SEQUENCE LISTING (2 sequences)

SEQ ID NO: 1 - protein, 481 aa, Arabidopsis thaliana
MKAEKVMEREIETTPIEPLSPMSHMLSSPNFFIVITFGFKTRCNRSAFVDGINNTLINAPRFSSKMEINYKKKGEPVWIPVKLRVDDHIIVPDLEYSNIQNPDQFVEDYTSNIANIPMDMSKPLWEFHLLNMKTSKAESLAIVKIHHSIGDGMSLMSLLLACSRKISDPDALVSNTTATKKPADSMAWWLFVGFWFMIRVTFTTIVEFSKLMLTVCFLEDTKNPLMGNPSDGFQSWKVVHRIISFEDVKLIKDTMNMKVNDVLLGMTQAGLSRYLSSKYDGSTAEKKKILEKLRVRGAVAINLRPATKIEDLADMMAKGSKCRWGNFIGTVIFPLWVKSEKDPLEYIRRAKATMDRKKISLEAFFFYGIIKFTLKFFGGKAVEAFGKRIFGHTSLAFSNVKGPDEEISFFHHPISYIAGSALVGAQALNIHFISYVDKIVINLAVDTTTIQDPNRLCDDMVEALEIIKSATQGEIFHKTEV

SEQ ID NO: 2 - DNA, 1446 bp, Arabidopsis thaliana
atgaaagcggaaaaagttatggagagagagatcgagacaacgccaatagagcctcttagcccaatgtcacatatgctaagttcgccaaatttcttcattgtcataacttttgggtttaaaactcgatgcaaccgatcggcctttgttgatggcatcaacaacacattgatcaatgctcccagattctccagtaaaatggagataaattataagaaaaaaggagaaccggtctggattcctgttaaacttcgagtagatgatcatattattgtgccggatcttgaatattccaacattcaaaatcctgatcagttcgtagaagactatacttcaaacatagctaatattccaatggacatgtccaaacctctttgggaatttcatttactcaacatgaagacatcaaaagcagaatctttggctatagtaaaaatacatcactccattggtgatgggatgtctcttatgtccctactacttgcttgttcacgaaaaatatcagaccctgacgcgctagtttctaataccacggctacaaaaaagccagctgattccatggcttggtggttgtttgttgggttttggtttatgataagagtcaccttcaccactattgttgagttttccaagttaatgcttacagtatgctttttggaagataccaaaaaccctcttatgggtaacccgagtgatggatttcaatcttggaaggttgtccatcggataataagttttgaggatgtcaagttaataaaggacacaatgaacatgaaggtgaatgatgttcttcttggaatgacacaagcaggtctttcaagatatttgagtagcaaatatgatggatcaacggctgagaagaaaaagatcttagaaaaactccgtgttcgtggtgccgtagctataaacttaagaccagcaacaaagatagaggatttggctgatatgatggcgaagggttcaaagtgtagatggggaaacttcataggaacggttatatttccgttgtgggtgaaatcagagaaagatccgttggagtacattcgacgagccaaagctacgatggataggaaaaaaatatctctggaagctttttttttctatggaattattaaattcaccttgaaattttttggaggaaaggcagtagaagcttttggaaagaggatatttggtcacacatcattggcattttcaaatgtgaaaggtcccgatgaagaaataagtttttttcaccatccgatttcatatatcgcaggaagtgcattagttggagcacaagctctcaatatccattttataagctacgtagacaagattgtcatcaatctagctgttgacacaacaacgattcaagatcctaatcgactatgtgatgatatggtggaagcacttgagatcatcaaatctgccacacaaggggaaatatttcacaaaacggaagtttga

(19) United States
(12) Patent Application Publication
(10) Pub. No.: US 2019/0161766 A1
(43) Pub. Date: May 30, 2019

(54) TRANSGENIC PLANT HAVING INCREASED EXPRESSION OF WAX SYNTHASE

(71) Applicant: The University of Massachusetts, Boston, MA (US)

(72) Inventors: Om Parkash Dhankher, Amherst, MA (US); Hesham M. Abdullah, Amherst, MA (US)

(21) Appl. No.: 16/193,008

(22) Filed: Nov. 16, 2018

Related U.S. Application Data
(60) Provisional application No. 62/591,855, filed on Nov. 29, 2017.

(51) Int. Cl. C12N 15/82; C12N 9/10; C12N 15/113; C12N 15/62

(52) U.S. Cl. CPC C12N 15/8273; C12N 9/1029; C12Y 203/0102; C12N 15/62; C12N 15/113

(57) ABSTRACT

Methods for producing plants with improved tolerance to stresses, such as drought or salinity, and transgenic plants made by the methods are disclosed. The methods comprise overexpressing a wax synthase/acyl-CoA:diacylglycerol acyltransferase (WSD1) in the plant.

### CROSS-REFERENCE TO RELATED APPLICATION

This application claims priority to U.S. provisional application No. 62/591,855, filed Nov. 29, 2017, the content of which is incorporated herein by reference in its entirety.

### SEQUENCE LISTING

A Sequence Listing, incorporated herein by reference, is submitted in electronic form as an ASCII text file, created Nov. 29, 2017, of size 6.3 KB, and named “7Y0668102.TXT”.

### BACKGROUND

Climate change has far-reaching implications for global food security and already substantially impacts agricultural production worldwide. As a result of changing climate, more extreme weather events such as elevated temperatures, drought, salinity, heavy metals contamination and other types of damaging weather are all expected to take tolls on crop yield and quality. World agriculture is facing a major crisis in order to produce enough foods for the growing world population. By 2050, farmers are expected to help increase global food production by 70-110% to feed a world population of more than 9 billion people, which is compounding with dramatic loss and degradation of arable land due to increasing salinity and drought.

The growing food demand and the threat of heavy crop losses due to global climate change make feeding the world's population in the future exceedingly challenging. Further, the Food and Agriculture Organization (FAO) of the United Nations estimated that agriculture consumes more than 70% of the fresh water, which is expected to be increase further in future. Under the current climate change scenario, a major constraint for crop production is availability of irrigation water and competition for water between urban and agricultural areas will compound the problem. Drought stress is expected to limit the productivity of more than half of the earth's arable land in the next 50 years, and competition for water between urban and agricultural areas will compound the problem.

Cuticular waxes are hydrophobic layer coatings in the epidermis of the aerial plant bodies that act as the first barrier between the plants and the surrounding environments. These waxes are varied in structures which ranged from cutin polyester to intra and epicuticular waxes (Lee LB and Suh MC (2015) Plant and Cell Physiology, 56(1):48 60). The chemical composition of cuticular waxes is also varied; they consist of solvent soluble lipids composed of very-long-chain fatty acids (VLCFAs) and their derivatives (including aldehydes, alkanes, fatty alcohols, ketones, and wax esters), with a small portion of phenylpropanoids and triterpenoids (Kunst L al. (2003) Prog Lipid Res 42: 51-80.). The chemical and physical properties of cuticular waxes render them useful in differential physiological functions. On plant surfaces, they can control non-stomatal water loss and gas exchange (Bernard, A. and Joubes, J. (2013) Prog. Lipid Res. 52: 110-129), provide protection against UV radiation (Lee and Suh, 2015), prevent the attachments of pathogen spores and atmospheric pollutants, in addition to their roles in controlling plant interactions with insects, fungal, and bacterial pathogens (Lee and Suh, 2015).

Cuticular wax biosynthetic pathways have been characterized initially in the model plant Arabidopsis thaliana, and many gene/enzymes involved in regulating those pathways are under study. (Suh MC et al. (2005) Plant Physiol. 139(4): 1649-1665; Lee and Suh, 2015). Cuticular waxes are synthesized in epidermal cells, and their synthesis is initiated by elongation of C16-C18 fatty acids, which are produced in plastids, to from VLCFAs (with carbon length up to >34) via the activity of fatty acid elongase (FAE) complex. FAE is comprised of four enzymes, β-ketoacyl-CoA synthase (KCS), β-ketoacyl-CoA reductase (KCR), β-hydroxyacyl-CoA dehydratase (HCD), and enoyl-CoA reductase (ECR), located in the endoplasmic reticulum (ER) (Lee and Suh, 2015). The elongated VLCFAs are subsequently activated into VLCFA-CoAs by the activity of a long chain acyl-CoA synthetase (LACS), and then biochemically modified via the alkane-forming pathway or primary alcohol-forming pathway (Kunst L and Samuels L. (2009) Current Opinion in Plant Biology, 12:721-727). In the alkane-forming pathway, active VLCFAs are converted into alkanes via the activity of aldehyde decarbonylase (protein ECERIFERUM, CER) and cytochrome b5 complexes (Bernard A et al. (2012) The Plant Cell, ;24(7):3106-3118). In the alcohol-forming pathway, the VLCFAs are reduced to primary alcohols via the activity of fatty acyl-CoA reductases, and the formed primary alcohols along with acyl-CoA pools with 16 carbon atoms, are subsequently esterified via the activity of bifunctional wax synthase/acyl-CoA:diacylglycerol acyltransferase (WSD1) (Rowland O et al (2006) Plant Physiology, 142(3):866-877; Lee SB et al. (2014) Plant Cell Rep. 33:1535-1546), As in Arabidopsis, several genes associated with cuticular wax biosynthesis have been identified and characterized in other crops, including maize, rice, and tomato (Hansen et al. (1997) Plant Physiol, 113:1091-1100; Xu et al. (1997) Plant Physiol 115 501-510; Jung et al. (2006) Plant Cell 18:3015-3032; Islam et al. (2009) Plant Mol Biol 70:443-456; Lee et al., 2014), reflecting the need for cuticular wax biosynthesis as a critical biological process in all plant species. However, the function and role of WSD1 and it's homologs in increasing cuticular wax and involvement in abiotic and biotic stresses is yet to characterized.

Plants usually exhibit various physiological and biochemical changes in response to abiotic stresses. These changes include stomata closure, reduction of photosynthesis efficiency and transpiration, and synthesis of stress-related secondary metabolites (Zingaretti et al. (2013). Water Stress and Agriculture, Responses of Organisms to Water Stress, Dr. Sener Akinci (Ed.), InTech, DOI: 10.5772/53877). Under drought stress conditions, plants have developed a strategy by which they can lessen the impact of drought stress by increasing the amounts of cuticular waxes. Under drought conditions, overexpression of Arabidopsis MYB96 reportedly activates expression of wax biosynthetic genes and increases accumulation of cuticular waxes (Seo et al. (2011) Plant Cell 23:1138-115).

To ensure global food security, there is a need in the art to develop climate-resilient crops that can thrive and produce better yields under extreme environmental conditions such as drought, salinity, high temperature, etc. and to develop generally applicable methods to develop such climate-resilient crops.

### SUMMARY

Disclosed, in various embodiments, are compositions and methods for producing plants with increased resistance to stress, such as drought, high salinity, high temperature, or a biotic stress.

A method of increasing stress-tolerance in a plant or in a part, cell, or propagation material thereof, wherein the stress is drought, high salinity, high temperature, or a biotic stress, comprises: expressing a transgenic expression cassette in a plant or in a tissue, organ, part, cell or propagation material thereof, wherein the transgenic expression cassette comprises a nucleic acid sequence encoding a wax synthase/acyl-CoA:diacylglycerol acyltransferase (WSD1) operatively linked to a plant-expressible promoter; and selecting the plant or the tissue, organ, part, cell or propagation material thereof in which expression of WSD1 is increased, cuticular wax content is increased in leaves or stem, water loss from leaves is reduced, or chlorophyll leaching is reduced in comparison with a plant of the same species, or a tissue, organ part, cell or propagation material thereof, that is not expressing the transgenic expression cassette.

A method of producing a transgenic plant having increased stress-tolerance, wherein the stress is drought, high salinity, or high temperature, the method comprises transforming a plant cell with a transgenic expression cassette comprising a nucleic acid sequence encoding a wax synthase/acyl-CoA:diacylglycerol acyltransferase (WSD1) operatively linked to a plant-expressible promoter; growing a plant from the transformed plant cell until the plant produces seed; and selecting a seed from a plant in which expression of WSD1 is increased, cuticular wax content is increased in leaves or stem, water loss from leaves is reduced, or chlorophyll leaching is reduced relative to a plant of the same species not comprising the transgenic expression cassette.

Also disclosed is a trangenic plant or a tissue, organ, part, cell, or propagation material thereof made by any of the disclosed methods.

A transgenic plant comprises transgenic expression cassette comprising a polynucleotide encoding a wax synthase/acyl-CoA:diacylglycerol acyltransferase (WSD1) operatively linked to a plant-expressible promoter.

A transgenic expression cassette comprising a polynucleotide encoding a wax synthase/acyl-CoA:diacylglycerol acyltransferase (WSD1) operatively linked to a plant-expressible promoter.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 illustrates the genetic transformation of A. thaliana and confirmation of AtWSD1 gene integration and expression into Arabidopsis shoots and roots. Panel (A) is a simplified diagram illustrated the T-DNA construct designed to manipulate cuticular waxes via constitutively overexpression of AtWSD1; Panel (B) is an image of results from PCR-genotyping to detect the presence of CaCMV-35S promoter fragment in the Arabidopsis genome; Panels (C) and (D) are bar charts of the results of qPCR analysis to detect the expression of AtWSD1 in Arabidopsis shoots (C) and roots (D) in a non-transgenic line, WT, and transgenic lines WSD #8-8, 8-12, 9-11, 9-12, 4-10, 4-11, 10-12.

FIG. 2 illustrates the regulation of the AtWSD1 gene under abiotic stresses. Two-week-old Arabidopsis plants were treated with stress conditions, including mannitol and NaCl, and the signaling hormone abscisic acid (ABA) at the indicated time points (0-24 hours). Mannitol treatment mimics drought stress in plants and ABA is a drought signalling molecule. Shown are the effects of mannitol on WSD1 gene expression in roots (A) and shoots (B); effects of NaCl on WSD1 gene expression in roots (C) and shoots (D); and effects of ABA on WSD1 gene expression in roots (E) and shoots (F). Transcript levels were examined via q-RT-PCR as indicated in the material and methods section.

FIG. 3 illustrates screening AtWSD1 transgenic lines for tolerance to ABA, mannitol, and salt stresses. WT (non-transgenic control plants and four AtWSD1 transgenic plants (WSD #4, 8, 9, or 10) were grown on ½ strength MS medium alone or supplemented with ABA (1 uM), mannitol (100 mM), or NaCl (75 mM) for 21 days.

FIG. 4 illustrates gain in root length and total plant biomass in Arabidopsis transgenics lines in response to stress conditions. A, C, E, and G represent root length (cm) measurements under control, ABA, mannitol, and NaCl treatments, respectively. B, D, F, and H represent plant total biomass (gm) under control, ABA, Mannitol, and NaCl treatments, respectively. Four biological replicates were averaged. Bars indicate standard error (SE) of the mean.

FIG. 5A presents photographs illustrating the phenotypic changes in four AtWSD1 transgenic lines (WSD #4, 8, 9, 10) and WT (Col-0) plants subjected to water withholding. Water was withheld from 4-wk-old Arabidopsis plants for 12 days before watering was resumed. Arabidopsis plants maintained under normal watering conditions (upper panel), 10 days after water withholding (middle panel), and 4 days after re-watering (lower panel).

FIG. 5B is a graph showing cumulative transpirational water loss from the five plant lines and FIG. 5C is a graph showing the results of chlorophyll leaching assays from desiccated Arabidopsis leaves of the WT and WSD1 overexpressing lines (WSD #4, 8, 9, 10).

Data in FIGS. 5B and 5C are represented as the mean of 5 independent measurements, with the bars representing the standard error (SE, n=5) of the mean.

FIG. 6 illustrates phenotypic changes in Arabidopsis transgenics and WT (Col-0) plants subjected to salt treatment, demonstrating that AtWSD1 transgenic plants exhibit improved salinity-stress tolerance in Arabidopsis. A. Images of 4-wk-old Arabidopsis WT and transgenic plants either un-treated (0 salt) or treated with water supplemented with 100 mM NaCl for 20 days. B. Images represent WT and transgenic plants treated with water supplemented with 100 mM NaCl for 20 days and recovered after watering with normal water for an additional 16 days.

FIG. 7 presents Scanning Electron Microscopy (SEM) images showing the effects of WSD1 overexpression on epicuticular wax deposition in Arabidopsis leaf and stem tissues. Four-week-old Arabidopsis plants expressing the CaMV35s::AtWSD1 gene cassette and wildtype (WT) grown in soil were examined for epicuticular wax crystals by SEM. Bars=2 um.

FIG. 8 illustrates cuticular wax loading and composition in AtWSD1 transgenic and WT plants obtained from Arabidopsis leaf tissues. Total wax loads and coverage (A) and carbon chain length (B) of individual compound classes are given with standard error (n=3). Rosette leaves of 4-week-old wild type (Col-0, WT) and WSD1 overexpressors grown in soil were used for analysis of cuticular wax loads. Three biological replicates were averaged and statistically analyzed using a student's t test (* denotes P<0.05).

FIG. 9 illustrates cuticular wax loading and composition in AtWSD1 transgenic and WT plants obtained from Arabidopsis stem tissues. Total wax loads and coverage (A) and carbon chain length (B) of individual compound classes are given with standard error (n=3). Stem segments of 4-week-old wild type (Col-0, WT) and WSD1 overexpressors grown in soil were used for analysis of cuticular wax loads. Three biological replicates were averaged and statistically analyzed using a student's t test (* denotes P<0.05). Bars indicate the mean values (ng per mg FW)±standard error (n=3).

### DETAILED DESCRIPTION

Transgenic plants having improved resistance to various abiotic and biotic stress conditions, and methods and expression cassettes for producing such transgenic plants are disclosed. The inventors have unexpectedly found that transgenic plants overexpressing the bifunctional wax synthase/acyl-CoA:diacylglycerol acyltransferase (WSD1) showed increased leaf and stem wax loading and altered composition of the waxes present. Overexpression of WSD1 in the transgenic plants resulted in significantly increased resistance of the plant to severe drought and salt stress conditions.

The disclosed transgenic plants, methods of making the plants, and transgenic expression cassettes for use in the methods are highly useful for enhancing the climate resilience of the plant by enabling the plants to produce better yields under various stress condition, including drought and salinity stress. Advantageously, the disclosed expression cassettes and methods for producing a transgenic plant with enhanced oil content and seed yield are applicable to a wide variety of plants, including any of the food, fiber, forage, or bioenergy crop plants. The overexpression of WSD1 in any of the food, fiber, forage, and bioenergy crop plants will enable the plants to produce better yields under various abiotic and biotic stress conditions, for example permitting develoment of climate resilient crops that can be grown with less water, and possibly even be irrigated with brackish or sea water.

Accordingly, methods of increasing increasing stress-tolerance in a plant or in a tissue, organ, part, cell, or propagation material thereof are disclosed.

In an embodiment, the method comprises: expressing a transgenic expression cassette in a plant or in a tissue, organ, part, cell or propagation material thereof, wherein the transgenic expression cassette comprises a nucleic acid sequence encoding a wax synthase/acyl-CoA:diacylglycerol acyltransferase (WSD1) operatively linked to a plant-expressible promoter; and selecting the plant or the tissue, organ, part, cell or propagation material thereof in which expression of WSD1 is increased, cuticular wax content is increased in leaves or stem, the water loss from leaves is reduced, or chlorophyll leaching is reduced in comparison with a plant of the same species, or a tissue, organ part, cell or propagation material thereof, that is not expressing the transgenic expression cassette.

In another aspect, methods of producing a transgenic plant having increased stress-tolerance are also disclosed.

In an embodiment, the method comprises transforming a plant cell with a transgenic expression cassette comprising a nucleic acid sequence encoding a wax synthase/acyl-CoA:diacylglycerol acyltransferase (WSD1) operatively linked to a plant-expressible promoter; growing a plant from the transformed plant cell until the plant produces seed; and selecting a seed from a plant in which the expression of WSD1 is increased, cuticular wax content is increased in leaves or stem, the water loss from leaves is reduced, or chlorophyll leaching is reduced relative to a plant of the same species not comprising the transgenic expression cassette.

In another aspect, a transgenic expression cassette is disclosed.

The transgenic expression cassette can comprise a polynucleotide encoding a wax synthase/acyl-CoA:diacylglycerol acyltransferase (WSD1) operatively linked to a plant-expressible promoter. The plant-expresible promoter is a constitutive promoter, for example the CaMV 35S promoter. The polynucleotide can be further operably linked to a transcription terminator, for example the actin 2 terminator.

In another aspect, a transgenic plant is disclosed. The transgenic plant has increased stress-tolerance.

The transgenic plant can be made by any of the methods disclosed herein.

In an embodiment, the transgenic plant comprises a polynucleotide encoding a wax synthase/acyl-CoA:diacylglycerol acyltransferase (WSD1) operatively linked to a plant-expressible promoter. The WSD1 is constitutively overexpressed in the transgenic plant.

The wax synthase/acyl-CoA:diacylglycerol acyltransferase (WSD1) can be from an organism of a genus selected from Arabidopsis, Tropaeolum, Brassica, soybean (Glycine max), Linum, Helianthus, Camelina, Arachis, Ricinus, Cuphea, Crambe, Avocado (Persea americana), wheat (Triticum), rice (Oryza), maize, foxtail (Setaria viridis), sorghum, barley, pearl millet, and Gossypium. Preferably, the WSD1 protein can be from an Arabidopsis plant. For example, the WSD1 can be from Arabidopsis thaliana (UniProtKB-Q93ZR6, SEQ ID NO:1; EnsemblPlants Gene AT5G37300, Transcript AT5G37300.1, SEQ ID NO:2).

Increased expression of WSD1 in the transgenic plant can be achieved by genome editing or mutation of the endogenous gene such that the edited or mutated endogenous gene has increased expression of the protein's activity compared to the unmodified endogenous gene, by increasing copy number of the endogenous gene, or by introducing a polynucleotide encoding at least one copy of a heterologous gene operably linked to a promoter expressible in the plant.

Herein, “cuticular wax content” or “wax loading” of the leaves or stems of a plant refers to the weight of cuticular wax extracted from a given weight of leaves or stems of the plant, for example by chloroform extraction using the method of Seo et al. 2011. The “cuticular wax content” or “wax loading” of the leaves or stems of a plant can be expressed as weight of the wax per unit weight of leaves or stems.

An increase in cuticular wax content in the leaves or stems of a transgenic plant made using the methods and constructs disclosed herein can be at least about, e.g. 1%, 2%, 3%, 4%, 5%, 6%, 7%, 8%, 9%, 10%, 1 1%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 105%, 110%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 296%, 300%, 310%, 320%, 330%, 340%, 350%, 375%, 400%, 450%, 500% or more, higher than the cuticular wax content of the leaves or stems of a control plant, i.e., a plant of the same species as the transgenic plant but that does not comprise the transgenic expression construct(s) disclosed herein. In particular embodiments, an increase in cuticular wax content of the leaves or stems of a transgenic plant disclosed herein can be an increase of at least about, e.g., 2% to about 100%, 5% to about 90%, about 5% to about 80%, about 5% to about 70%, about 10% to about 90%, about 10% to about 80%, about 10% to about 70%, about 10% to about 60%, about 10% to about 50%,about 10% to about 740%, about 15% to about 80%, about 15% to about 75%, about 15% to about 50%, about 15% to about 40%, about 15% to about 30%, about 20% to about 80%, about 20% to about 75%, about 20% to about 70%, about 20% to about 60%, about 30% to about 80%, about 30% to about 75%, and the like compared to the cuticular wax content of the leaves or stems of a control plant.

Herein, “water loss” from the leaves of a plant refers to the weight of leaves of the plant, determined using the water loss assay method of Seo et al. 2011. The “water loss” of the leaves of a plant can be expressed as dried leaf weight at the indicated time relative to the initial fresh weight, or as a percent thereof.

The water loss from the leaves of the transgenic plant at one hour can be no more than 40%, no more than 50%, no more than 55%, no more than 60%, no more than 65%, no more than 70%, no more than 75%, no more than 80%, no more than 85% of that of the initial fresh weight.

A reduction in water loss at one hour from leaves of a transgenic plant disclosed herein can be at least 5%, at least 10%, at least 15%, at least 20%, at least 25%, at least 30%, at least 35%, at least 40%, at least 45%, at least 50% , at least 55% reduced from the water loss at one hour from leaves of a control plant.

Herein, “chlorophyll leaching” from the leaves of a plant refers to the amount of chlorphyl extracted from leaves of the plant, determined using the chlorophyll leachings assay of Seo et al. 2011. The “chlorophyll leaching” of the leaves of a plant can be expressed as a percent chlorophyll extracted relative to that extracted at 24 h after initial immersion in ethanol.

The chlorphyll leaching from the leaves of the transgenic plant at one hour after initial immersion in ethanol can be no more than 30%, no more than 35%, no more than 40%, no more than 45%, no more than 50%, no more than 55%, no more than 60% of that extracted at 24 h after initial immersion in ethanoht.

A reduction in chloroplyll leaching from leaves of a transgenic plant disclosed herein at one hour after initial immersion in ethanol can be at least 5%, at least 10%, at least 15%, at least 20%, at least 25%, at least 30%, at least 35%, at least 40%, at least 45%, at least 50% , at least 55% reduced from the chloroplyll leaching from leaves of a control plant at one hour after initial immersion in ethanol.

Herein, “plant” refers to all genera and species of higher and lower plants of the Plant Kingdom. The term includes the mature plants, seeds, shoots and seedlings, and parts, propagation material, plant organ tissue, protoplasts, callus and other cultures, for example cell cultures, derived from them, and all other species of groups of plant cells giving functional or structural units. Mature plants refers to plants at any developmental stage beyond the seedling. Seedling refers to a young, immature-plant at an early developmental stage.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell.

“Plant” encompasses all annual and perennial monocotyldedonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, pearl millet, Setaria, Secale, Triticum, Sorghum, Picea and Populus.

Preferred plants are those from the following plant families: Amaranthaceae, Asteraceae, Brassicaceae, Carophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanaceae, Sterculiaceae, Tetragoniaceae, Theaceae, Umbelliferae, Poaceae (formerly Gramineae).

The invention can particularly be applied advantageously to dicotyledonous plant organisms. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the specis napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit.

Also of interest for transformation are plants which are oil crop plants, also referred to as “oilseed plants” herein. Oil crop plants are understood as being plants whose oil content is naturally high and/or which can be used for the industrial production of oils. These plants can have a high oil content and/or else a particular fatty acid composition which is of interest industrially. Preferred plants are those with a lipid content of at least 1% by weight. Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Arachis hypogea (groundnut); Helianthus annuus (sunflower); Linum usitatissimum (linseed or flax); Oenothera biennis (evening primrose); Olea europaea (olive); Oryza sativa (rice); Ricinus communis (castor); Sesamum indicum (sesame); Triticum species (wheat); Setaria species (Setaria viridis), pearl millet (Pennisetum glaucum), Zea mays (maize), and various nut species such as, for example, walnut or almond.

Camelina species, commonly known as false flax, are native to Mediterranean regions of Europe and Asia and seem to be particularly adapted to cold semiarid climate zones (steppes and prairies). The species Camelina sativa was historically cultivated as an oilseed crop to produce vegetable oil and animal feed. It has been introduced to the high plain regions of Canada and parts of the United States as an industrial oilseed crop. As a result of its high oil content (˜35%) of its seeds, its frost tolerance, short production cycle (85-100 days), and insect resistance, it is an interesting target as a source for production of biofuels.

The terms “polypeptide,” “peptide” and “protein” are used interchangeably herein to refer to a molecule formed from the linking, in a defined order, of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis.

The term “nucleic acid”, “polynucleotide”, or “oligonucleotide” includes DNA molecules and RNA molecules. A polynucleotide may be single-stranded or double-stranded. A polynucleotide can be obtained by a suitable method known in the art, including isolation from natural sources, chemical synthesis, or enzymatic synthesis. Nucleotides may be referred to by their commonly accepte single-letter codes.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). In some embodiments, gene refers to a coding sequence operably linked to a promoter.

“Homolog” is a term used in the art to indicate a polynucleotide or polypeptide sequence possessing a high degree of sequence relatedness to a subject sequence. Such relatedness may be quantified by determining the degree of identity and/or similarity between the sequences being compared. Falling within this generic term are the terms “ortholog” meaning a polynucleotide or polypeptide that is the functional equivalent of a polynucleotide or polypeptide in another species, and “paralog” meaning a functionally similar sequence when considered within the same species. Paralogs present in the same species or orthologs of a gene in other species can readily be identified without undue experimentation, by molecular biological techniques well known in the art. As used herein, WSD1 refers to WSD1 as well as its homologs and orthologs.

Related polypeptides to a reference polypeptide (for example, WSD1) are aligned with the reference polypeptide by assigning degrees of homology to various deletions, substitutions and other modifications. Homology can be determined along the entire polypeptide or polynucleotide, or along subsets of contiguous residues. The percent identity is the percentage of amino acids or nucleotides that are identical when the two sequences are compared. The percent similarity is the percentage of amino acids or nucleotides that are chemically similar when the two sequences are compared. A reference polypeptide and homologous polypeptides of the reference polypeptide are preferably greater than or equal to about 75%, preferably greater than or equal to about 80%, more preferably greater than or equal to about 90% or most preferably greater than or equal to about 95% identical.

In addition, polynucleotides that are substantially identical to a polynucleotide encoding a WSD1 polypeptide are included. By “substantially identical” is meant a polypeptide or polynucleotide having a sequence that is at least about 85%, specifically about 90%, and more specifically about 95% or more identical to the sequence of the reference amino acid or nucleic acid sequence.

Reference herein to either the nucleotide or amino acid sequence of WSD1 also includes reference to naturally occurring variants of these sequences. Non-naturally occurring variants that differ from the nucleotide or amino acid sequence of WSD1 and retain biological function are also included herein. For example, non-naturally occurring polypeptide variants that differ from the polypeptide encoded bySEQ ID NO:1 and retain biological function are also included herein. Preferably the variants comprise those polypeptides having conservative amino acid changes, i.e., changes of similarly charged or uncharged amino acids. Genetically encoded amino acids are generally divided into four families: (1) acidic (aspartate, glutamate); (2) basic (lysine, arginine, histidine); (3) non-polar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. As each member of a family has similar physical and chemical properties as the other members of the same family, it is reasonable to expect that an isolated replacement of a leucine with an isoleucine or valine, an aspartate with a glutamate, a threonine with a serine, or a similar replacement of an amino acid with a structurally related amino acid will not have a major effect on the binding properties of the resulting molecule. Variants of polypeptidescan be made by methods known in the art, for example, site-directed mutagenesis of polynucleotides, by random mutation, by chemical synthesis, or by chemical or enzymatic cleavage of the polypeptides. Whether an amino acid change results in a functional polypeptide can be determined by assaying the properties of transgenic plants containing the variant of WSD1.

A “transgenic plant” is one that has been genetically modified to contain and express recombinant DNA sequences, either as regulatory RNA molecules or as proteins. As specifically exemplified herein, a transgenic plant is genetically modified to contain and express a recombinant DNA sequence operatively linked to and under the regulatory control of transcriptional control sequences that function in plant cells or tissue or in whole plants. As used herein, a transgenic plant also encompasses progeny of the initial transgenic plant where those progeny contain and are capable of expressing the recombinant coding sequence under the regulatory control of the plant-expressible transcription control sequences described herein. Seeds containing transgenic embryos are encompassed within this definition.

Individual plants within a population of transgenic plants that express a recombinant gene may have different levels of gene expression. The variable gene expression is due to multiple factors including multiple copies of the recombinant gene, chromatin effects, and gene suppression. Accordingly, a phenotype of the transgenic plant may be measured as a percentage of individual plants within a population. In one embodiment, greater than or equal to about 25% of the transgenic plants express the phenotype. Specifically, greater than or equal to about 50% of the transgenic plants express the phenotype. More specifically, greater than or equal to about 75% of the transgenic plants express the phenotype. The phenotype is preferably increased oil content of the plant or seeds of the plant or increased seed yield of the plant.

The transgenic plant has been transformed with an expression cassette comprising a protein or functional nucleic acid coding sequence operatively linked to a plant-expressible transcription regulatory sequence.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A transgenic plant can be produced by selection of transformed seeds or by selection of transformed plant cells and subsequent regeneration.

The coding sequence of interest, for example a coding sequence for a WSD1 polypeptide, can be subcloned under the control of a promoter, such as the CaMV35S promoter, and the actin 2 terminator into a plant expression binary vector such as pCAMBIA-RedSeed, which expresses the DsRed fluorescent protein under a seed-specific napin promoter for identification of transformants by red fluorescence in seeds. The vector pCAMBIA-RedSeed is described in U.S. Patent Application Publication No. 2018/0237792 A1.

Camelina sativa can be transformed using vacuum infiltration technology, and the T1 generation seeds are screened for BASTA resistance and/or red fluorescence. Transgenic plants transformed with the heterologous polynucleotide are produced. Tthe plant can also express one or more additional heterologous coding sequences.

The transgenic plants are grown (e.g., on soil) and harvested. In one embodiment, above ground tissue is harvested separately from below ground tissue. Suitable above ground tissues include shoots, stems, leaves, flowers, grain, and seed. Exemplary below ground tissues include roots and root hairs. In one embodiment, whole plants are harvested and the above ground tissue is subsequently separated from the below ground tissue.

The terms “increase,” “increasing,” “increased,” “enhance,” “enhanced,” “enhancing,” and “enhancement” (and grammatical variations thereof), as used herein, describe an elevation in gene expression or a phenotypic trait in a plant, plant cell and/or plant part as compared to a control as described herein. This increase can be observed by comparing the increase in expression or the phenotypic trait in the plant, plant part or plant cell transformed with, for example, one or more expression cassettes disclosed herein to the appropriate control (e.g., the same plant, plant part, and/or plant cell lacking (i.e., not transformed with) the one or more heterologous polynucleotides).

As used herein, the terms “reduce,” “reduced,” “reducing,” “reduction,” “diminish,” “suppress,” and “decrease” (and grammatical variations thereof), describe, for example, a decrease in gene expression or a phenotypic trait in a plant, plant cell and/or plant part as compared to a control as described herein.

As used herein, the terms “express,” “expression,” and the like, with respect to a nucleotide sequence (e.g., RNA or DNA) indicates that the nucleotide sequence is transcribed and, optionally, translated. Thus, a nucleotide sequence may express a polypeptide of interest or a functional untranslated RNA. A “functional” RNA includes any untranslated RNA that has a biological function in a cell, e.g., regulation of gene expression. Such functional RNAs include but are not limited to RNAi (e.g., siRNA, shRNA, antisense RNA), miRNA, ribozymes, RNA aptamers, and the like.

As used herein, the term “overexpression” means increased expression over that in the control. In some embodiments, “overexpression” can include expression of a heterologous polynucleotide not normally expressed in an organism. In other embodiments, overexpression can include expression of an endogenous polynucleotide in a transgenic expression cassette such that the amount of the endogenous polypeptide produced as a result of the endogenous polynucleotide in the transgenic expression cassette is greater than is produced in the organism not transformed with the expression cassette.

IThe WSD1 gene can be expressed in an expression vector suitable for in vivo expression such as, for example, plant expression systems. The expression cassette for the WD1 gene can be inserted into a recombinant expression vector or vectors.

The term “expression vector” or “vector” refers to a plasmid, virus, or other means known in the art that has been manipulated by insertion or incorporation of a genetic sequence of interest. The term “plasmids” generally is designated herein by a lower case p preceded and/or followed by capital letters and/or numbers, in accordance with standard naming conventions that are familiar to those of skill in the art. Plasmids disclosed herein are either commercially available, publicly available on an unrestricted basis, or can be constructed from available plasmids by routine application of well-known, published procedures. Many plasmids and other cloning and expression vectors are well known and readily available, or those of ordinary skill in the art may readily construct any number of other plasmids suitable for use. These vectors are transformed into a suitable host cell to form a host cell vector system for the production of a polypeptide.

As used herein, “expression cassette” or “transgenic expression cassette” means a recombinant nucleic acid molecule comprising at least one coding sequence of interest operably linked with at least a control sequence (e.g., a promoter). The term “recombinant polynucleotide” refers to a polynucleotide that is made by the combination of two otherwise separated segments of sequence accomplished by the artificial manipulation of isolated segments of polynucleotides by genetic engineering techniques or by chemical synthesis. The coding sequence of interest can be, e.g., a polynucleotide encoding a WSD1 gene. Thus, transgenic expression cassettes designed to express a polynucleotide encoding a WSD1 gene are disclosed herein.

An expression cassette comprising a recombinant nucleic acid molecule may be chimeric, meaning that at least one of its components is heterologous with respect to at least one of its other components. In some embodiments, the expression cassettes comprising the heterologous polynucleotides can comprise one or more regulatory elements in addition to a promoter as described herein (e.g., enhancers, introns, translation leader sequences, translation termination sequences, and polyadenylation signal sequences). However, a transgenic expression cassette is also understood as meaning those constructs where a nucleic acid sequence encoding a nonendogenous polypeptide is placed behind an endogenous plant promoter in such a way that the latter brings about the expression of the nonendogenous polypeptide.

Operable linkage and a transgenic expression cassette can both be effected by means of conventional recombination and cloning techniques as they are described, for example, in Maniatis T, Fritsch E F and Sambrook J (1989) Molecular Cloning: A Laboratory Manual, Cold Spring Harbor Laboratory, Cold Spring Harbor (N.Y.), in Silhavy T J, Berman M L and Enquist L W (1984) Experiments with Gene Fusions, Cold Spring Harbor Laboratory, Cold Spring Harbor (N.Y.), in Ausubel F M et al. (1987) Current Protocols in Molecular Biology, Greene Publishing Assoc. and Wiley Interscience and in Gelvin et al. (1990) In: Plant Molecular Biology Manual. However, further sequences which, for example, act as a linker with specific cleavage sites for restriction enzymes, or of a signal peptide, may also be positioned between the two sequences. Also, the insertion of sequences may lead to the expression of fusion proteins. Preferably, the expression cassette composed of a promoter linked to a nucleic acid sequence to be expressed can be in a vector-integrated form and can be inserted into a plant genome, for example by transformation.

Furthermore, such polynucleotides can be stacked with any combination of nucleotide sequences to create plants, plant parts, and/or plant cells having the desired phenotype. Stacked combinations can be created by any method including, but not limited to, any conventional methodology (e.g., cross breeding for plants), or by genetic transformation. If stacked by genetic transformation, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by a single promoter or by separate promoters, which can be the same or different, or a combination thereof. It is further recognized that nucleotide sequences can be stacked at a desired genomic location using a site-specific recombination system. See, e.g. , Int'l Patent Application Publication Nos. WO 99/25821; WO 99/25854; WO 99/25840; WO 99/25855 and WO 99/25853.

The term “transgene” refers to a recombinant polynucleotide or nucleic acid that comprises a coding sequence encoding a protein or RNA molecule.

The transgenic expression cassettes are inserted into a vector adapted for expression in a plant, bacterial, yeast, insect, amphibian, or mammalian cell that further comprises the regulatory elements necessary for expression of the nucleic acid molecule in the plant, bacterial, yeast, insect, amphibian, or mammalian cell operatively linked to the nucleic acid molecule encoding a sequence of interest for expression.

“Operatively linked” or “operably linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. For instance, a promoter is operatively linked with a nucleotide sequence if the promoter effects the transcription or expression of the nucleotide sequence. Those skilled in the art will appreciate that the control sequences (e.g., promoter) need not be contiguous with the nucleotide sequence to which it is operably linked, as long as the control sequences function to direct the expression thereof. Thus, for example, intervening untranslated, yet transcribed, sequences can be present between a promoter and a nucleotide sequence, and the promoter can still be considered “operatively linked” to the nucleotide sequence. Expression control sequences such as, for example, enhancer sequences can also exert their function on the target sequence from positions which are further removed or indeed from other DNA molecules.

As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns (if introns are present), translation leader sequences, translation termination sequences, polyadenylation signal sequences, and stop codons. The term “control sequences” is intended to include, at a minimum, components whose presence can influence expression, and can also include additional components whose presence is advantageous, for example, leader sequences and fusion partner sequences. Expression control sequences can include a promoter. By “promoter” is meant minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included. If a promoter is inducible, there are sequences present that mediate regulation of expression so that the associated sequence is transcribed only when an inducer (e.g., light or an exogenous chemical regulator) is available to the plant or plant tissue. An exemplary promoter to provide basal expression in transgenic plants is the 35S cauliflower mosaic virus (CaMV) promoter.

Any promoter useful for initiation of transcription in a cell of a plant can be used in the expression cassettes of the present invention. Promoters can include, for example, constitutive, inducible, temporally regulated, developmentally regulated, chemically regulated, and/or tissue-specific promoters for use in the preparation of recombinant nucleic acid molecules, for example transgenic expression cassettes or recombinant polynucleotides. These various types of promoters are known in the art. Promoters can be identified in and isolated from the plant to be transformed and then inserted into the expression cassette to be used in transformation of the plant. Alternatively, nonendogenous promoters isolated from other plants, but functional in the plant to be transformed, can be inserted into the expression cassettes.

Promoters which are preferably introduced into the transgenic expression cassettes are those which are operable in a plant or a tissue, organ, part, cell or propagation material of the plant. A promoter which is operable in plants is understood as meaning any promoter which is capable of governing the expression of genes, in particular nonendogenous genes, in plants or plant parts, plant cells, plant tissues or plant cultures.

A “constitutive” promoter refers to a-promoter which ensures expression in a large number of, preferably all, tissues over a substantial period of plant development, preferably at all times during plant development (Benfey et al.(1989) EMBO J 8:2195-2202). A plant promoter or promoter originating from a plant virus is preferably used. The promoter of the CaMV (cauliflower mosaic virus) 35S transcript (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228) or the 19S CaMV promoter (U.S. Pat. No. 5,352,605; WO 84/02913; Benfey et al. (1989) EMBO J 8:2195-2202) are preferred. Another suitable constitutive promoter is the Rubisco small subunit (SSU) promoter (U.S. Pat. No. 4,962,028), the leguminB promoter (GenBank Acc. No. X03677), the promoter of the nopalin synthase from Agrobacterium, the TR dual promoter, the OCS (octopine synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of the vacuolar ATPase subunits, and the promoter of the Arabidopsis thaliana nitrilase-1 gene (GenBank Acc. No.: U38846, nucleotides 3862 to 5325 or else 5342), and further promoters of genes whose constitutive expression in plants is known to the skilled worker. Additional examples include cestrum virus promoter (cmp) (U.S. Pat. No. 7,166,770), nos promoter (Ebert et al. (1987) Proc. Natl. Acad. Sci USA 84:5745-5749), Adh promoter (Walker et al. (1987) Proc. Natl. Acad, Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94) and arabidopsis (Norris et al. 1993. Plant Molec. Biol. 21 :895-906).

A “tissue-specific promoter” refers to a-promoter which ensures expression in a specific tissue over a substantial period of plant development. A preferred tissue for localization of expression is a seed. Examples of seed-specific promoters include the phaseolin promoter (U.S. Pat. No. 5,504,200; Bustos M M et al. (1989) Plant Cell 1.(9):839-53), the promoter of the 2S albumin gene (Joseffson L G et al. (1987) J Biol Chem 262:12196-12201), the legumine promoter (Shirsat A et al. (1989) Mol Gen Genet 215(2):326-331), the USP (unknown seed protein) promoter (Bäumlein H et al. (1991) Mol Gen Genet 225(3):459-67), the napin gene promoter (U.S. Pat. No. 5,608,152; Stalberg K et al. (1996) L Planta 199:515-519), the promoter of the sucrose binding proteins (WO 00/26388) or the legumin B4 promoter (LeB4; Bäumlein H et al. (1991) Mol Gen Genet 225: 121-128; Bäumlein et al. (1992) Plant Journal 2(2):233-9; Fiedler U et al. (1995) Biotechnology (NY) 13(10);:1090f), the Arabidopsis oleosin promoter (WO 98/45461), and the Brassica Bce4 promoter (Wo 91/13980). Further suitable seed-specific promoters are those of the gene encoding high-molecular weight glutenin (HMWG), gliadin, branching enyzme, ADP glucose pyrophosphatase (AGPase), starch synthase, or glycinin. Yet other examples include promoters associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1). Also useful for seed-specific expression is the pea vicilin promoter (Czako et al. (1992) Mol. Gen. Genet. 235:33-40; as well as the seed-specific promoters disclosed in U.S. Pat. No. 5,625,136.

The expression cassettes may also contain a “chemically-inducible promoter” (review article: Gatz et al. (1997) Annu Rev Plant Physiol Plant Mol Biol 48:89-108), by means of which the expression of an exogenous gene in the plant can be controlled at a particular point in time. Such promoters include, for example, the PRP1 promoter (Ward et al. (1993) Plant Mol Biol 22:361-366), a salicylic acid-inducible promoter (WO 95/19443), a benzenesulfonamide-inducible promoter (EP 0 388 186), a tetracyclin-inducible promoter (Gatz et al. (1992) Plant J 2:397-404), an abscisic acid-inducible promoter EP 0 335 528), and an ethanol-cyclohexanone-inducible promoter (WO 93/21334). Also suitable is the promoter of the glutathione-S transferase isoform II gene (GST-II-27), which can be activated by exogenously applied safeners such as, for example, N,N-diallyl-2,2-dichloroacetamide (WO 93/01294) and which is operable in a large number of tissues of both monocots and dicots.

In addition, further promoters which make possible expression in further plant tissues or in other organisms such as, for example, E. coli or yeast, may be linked operably with the nucleic acid sequence-to be expressed.

“Plant-expressible transcriptional and translational regulatory sequences” are those that can function in plants, plant tissue and/or plant cells to effect the transcriptional and translational expression of the nucleotide sequences with that they are associated. Included are 5′ sequences that qualitatively control gene expression (turn on or off gene expression in response to environmental signals such as light, or in a tissue-specific manner) and quantitative regulatory sequences that advantageously increase the level of downstream gene expression. An example of a sequence motif that serves as a translational control sequence is that of the ribosome binding site sequence. Polyadenylation signals are examples of transcription regulatory sequences positioned downstream of a target sequence. Exemplary flanking sequences include the 3′ flanking sequences of the nos gene of the Agrobacterium tumefaciens Ti plasmid. The upstream nontranslated sequence of a bacterial merA coding sequence can be utilized to improve expression of other sequences in plants as well.

An expression cassette, or recombinant polynucleotide, encoding a WSD1 polypeptide operably linked to a promoter can be introduced into a plant in any combination with one or more additional polynucleotides to increase the cuticular wax content in a plant.

In some embodiments of the invention, an expression vector or cassette can comprise an enhancer sequence. Enhancer sequences can be derived from, for example, any intron from any highly expressed gene. In particular embodiments, an enhancer sequence usable in an expression vector or cassette disclosed herein includes, but is not limited to, the nucleotide sequence of ggagg (e.g., ribosome binding site).

An expression cassette or vector also can optionally include a transcriptional and/or translational termination region (i.e., termination region) that is functional in plants, yeast, or bacteria. A variety of transcriptional terminators is available for use in expression cassettes or vectors disclosed herein. The termination region may be native to the transcriptional initiation region, may be native to the operably linked nucleotide sequence of interest, may be native to the host cell, or may be derived from another source (i.e., foreign or heterologous to the promoter, the nucleotide sequence of interest, the host cell, or any combination thereof). Non-limiting examples of transcriptional terminators useful for plants can be a CAMV 35S terminator, a tml terminator, a nopaline synthase terminator and/or a pea rbcs E9 terminator, a RubisCo small subunit gene 1 (TrbcS1) terminator, an actin gene (Tactin) terminator, a nitrate reductase gene (Tnr) terminator, and/or aa duplicated carbonic anhydrase gene 1 (Tdcal) terminator.

The choice of vector used for constructing a recombinant DNA molecule depends on the functional properties desired, e.g., replication, protein expression, and the host cell to be transformed. In one embodiment, the vector comprises a prokaryotic replicon, i.e., a DNA sequence having the ability to direct autonomous replication and maintenance of the recombinant DNA molecule extra-chromosomally when introduced into a prokaryotic host cell, such as a bacterial host cell. In addition, the vector may also comprise a gene whose expression confers a selective advantage, such as a drug resistance, to the bacterial host cell when introduced into those transformed cells. Suitable bacterial drug resistance genes are those that confer resistance to ampicillin or tetracycline, among other selective agents. The neomycin phosphotransferase gene has the advantage that it is expressed in eukaryotic as well as prokaryotic cells.

Vectors typically include convenient restriction sites for insertion of a recombinant DNA molecule. Suitable vector plasmids include pUC8, pUC9, pBR322, and pBR329 available from BioRad Laboratories (Richmond, Calif) and pPL, pK and K223 available from Pharmacia (Piscataway, N.J.), and pBLUESCRIPT® and pBS available from Stratagene (La Jolla, Calif.). Other exemplary vectors include pCMU. Other appropriate vectors may also be synthesized, according to known methods; for example, vectors pCMU/Kb and pCMUII which are modifications of pCMUIV.

Suitable expression vectors capable of expressing a recombinant nucleic acid sequence in plant cells and capable of directing stable integration within the host plant cell include vectors derived from the tumor-inducing (Ti) plasmid of Agrobacterium tumefaciens, and several other expression vector systems known to function in plants. See for example, Verma et al., No. WO87/00551, incorporated herein by reference. Other suitable expression vectors include gateway cloning-compatible plant destination vectors for expression of proteins in transgenic plants, e.g., the pEarleygate series (Earley et al. The Plant Journal Volume 45, Issue 4, pages 616-629, February 2006) or gateway pDONR entry vectors.

Expression cassettes and expression vectors optionally contain a selectable marker, which can be used to select a transformed plant, plant part, and/or host cell. As used herein, “selectable marker” means a nucleotide sequence that when expressed imparts a distinct phenotype to a plant, plant part and/or cell expressing the marker and thus allows such a transformed plant, plant part, and/or cell to be distinguished from that which does not have the marker. Such a nucleotide sequence may encode either a selectable or screenable marker, depending on whether the marker confers a trait that can be selected for by chemical means, such as by using a selective agent (e.g., an antibiotic, herbicide, or the like), or whether the marker is simply a trait that one can identify through observation or testing, such as by screening (e.g., the R-locus trait). Of course, many examples of suitable selectable markers are known in the art and can be used in the expression cassettes described herein. Although such a marker gene may be carried on another polynucleotide sequence co-introduced into the host cell, it is most often contained on the cloning vector. Only those host cells into which the marker gene has been introduced will survive and/or grow under selective conditions. Suitable selection genes encode proteins that (a) confer resistance to antibiotics or other toxic substances, e.g., ampicillin, neomycin, methotrexate, etc.; (b) complement auxotrophic deficiencies; or (c) supply critical nutrients not available from complex media. The choice of the proper selectable marker will depend, in part, on the host cell.

Examples of selectable markers include, but are not limited to, a nucleotide sequence encoding aadA (i.e., spectinomycin and streptomycin resistance), a nucleotide sequence encoding neo (i.e., kanamycin resistance), a nucleotide sequence encoding aphA6 (i.e., kanamycin resistance), a nucleotide sequence encoding nptll (i.e., kanamycin resistance), a nucleotide sequence encoding bar (i.e., phosphinothricin resistance), a nucleotide sequence encoding cat (i.e., chloramphenicol resistance), a nucleotide sequence encoding badh (i.e., betaine aldehyde resistance), a nucleotide sequence encoding egfp, (i.e., enhanced green fluorescence protein), a nucleotide sequence encoding gfp (i.e., green fluorescent protein), a nucleotide sequence encoding a red fluorescent protein (e.g., mCherry or DsRed) a nucleotide sequence encoding luc (i.e., luciferase), a nucleotide sequence encoding ble (bleomycin resistance), a nucleotide sequence encoding ereA (erythromycin resistance), and any combination thereof.

Further examples of selectable markers useful with the invention include, but are not limited to, a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al, “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known {e.g., PAD AC, a chromogenic cephalosporin) (Sutcliffe (1978) Proc. Natl. Acad, Sci. USA 75:3737-3741); a nucleotide sequence encoding xylE that encodes a catechol dioxygenase (Zukowsky et al. (1983) Proc. Natl. Acad. Sex. USA 80:1101-1105); a nucleotide sequence encoding tyrosinase, an enzyme capable of oxidizing tyrosine to DOPA and dopaquinone, which in turn condenses to form melanin (Katz et al. (1983) J Gen. Microbiol. 129:2″/'03-21′I 4); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding Bla that confers ampicillin resistance; or a nucleotide sequence encoding aequorin which may be employed in calcium-sensitive bioluminescence detection (Prasher et al. (1985) Biochem. Biophys. Res. Comm. 126:1259-1268), and/or any combination thereof. One of skill in the art is capable of choosing a suitable selectable marker for use in an expression cassette of this invention.

One of the most commonly used markers for the selection of transgenic plants is resistance to glufosinate ammonium, an herbicide that is sold under a variety of trade names including Basta and Finale. Resistance to glufosinate ammonium is conferred by the bacterial bialophos resistance gene (BAR) encoding the enzyme phosphinotricin acetyl transferase (PAT). The major advantage of glufosinate ammonium selection is that it can be performed on plants growing in soil and does not require the use of sterile techniques.

Transformation of a host cell with an expression vector or other DNA is carried out by conventional techniques known in the art. By “transformation” is meant a permanent or transient genetic change induced in a cell following incorporation of new DNA (i.e., DNA exogenous to the cell). Where the cell is a plant cell, a permanent genetic change is generally achieved by introduction of the DNA into the genome of the cell. By “transformed cell” or “host cell” is meant a cell (e.g., prokaryotic or eukaryotic) into which (or into an ancestor of which) has been introduced, by means of recombinant DNA techniques, a DNA molecule encoding a WSD1 polypeptide.

The method of transformation in obtaining such transgenic plants is not critical, and various methods of plant transformation are currently available. Furthermore, as newer methods become available to transform crops, they may also be directly applied hereunder. For example, many plant species naturally susceptible to Agrobacterium infection may be successfully transformed via tripartite or binary vector methods of Agrobacterium mediated transformation. In many instances, it will be desirable to have the construct bordered on one or both sides by T-DNA, particularly having the left and right borders, more particularly the right border. This is particularly useful when the construct uses A. tumefaciens or A. rhizogenes as a mode for transformation, although the T-DNA borders may find use with other modes of transformation. In addition, techniques of microinjection, DNA particle bombardment, and electroporation have been developed which allow for the transformation of various monocot and dicot plant species.

In addition to transformation technology, traditional breeding methods as known in the art (e.g., crossing) can be used to assist in introducing into a single plant each of the expression cassettes described herein o produce a plant, plant part, and/or plant cell comprising and expressing each of the recombinant polynucleotides as described herein.

Recombinant host cells, in the present context, are those that have been genetically modified to contain a heterologous DNA molecule. The DNA can be introduced by a means that is appropriate for the particular type of cell, including without limitation, transfection, transformation, lipofection, or electroporation.

The compositions and methods disclosed herein are further illustrated by the following non-limiting examples, which are merely illustrative and are not intended to limit the scope hereof. Any variations in the exemplified compositions and methods that occur to the skilled artisan are intended to fall within the scope of the present invention.

### EXAMPLES

### Example 1

### Engineering Camelina and Arabidopsis Transgenics Over-Expressing the Arabidopsis Wax Synthase (WSD1) Gene

In this example, the Arabidopsis wax synthase (WSD1) gene was constitutively overexpressed in Arabidopsis and Camelina under the control of the CaMV35S promoter. Transgenic lines were generated and tested for increased deposition of epicuticular wax crystals and wax load in leaf and stem surfaces, which can contribute to enhanced tolerance to drought.

### Materials and Methods

### Generation of Arabidopsis and Camelina Transgenics

Camelina and Arabidopsis transgenic lines were generated by introducing the Arabidopsis WSD1 gene using gene cassette CaMV35S-p:WSD1:Actin2-t. This cassette was cloned into the plant expression vector pCAMBIA-RedSeed, described in U.S. Patent Application Ser. No. 62/462,624, and used to transform both Camelina and Arabidopsis using Agrobacterium-mediated flower-dip method as described in Lu and Kang (Plant Cell Rep, 2008, 27:273-278). Camelina and Arabidopsis plants at maturity were harvested. The T1 seeds of Camelina were selected for the expression of DsRed fluorescence, while the T1 seeds of Arabidopsis were selected for hygromycin antibiotic resistance on hygromycin-containing medium.

PCR Genotyping and qRT-PCR for Gene Expression Analysis

To confirm the successful introduction of WSD1 gene cassette into Camelina and Arabidopsis genome, gDNA was isolated from leaf tissues using the cetyl trimethylammonium bromide (CTAB) method and was used as a template for PCR reaction to amplify the CaMV35S promoter fragment. To measure the expression level of WSD1 gene in the transgenic and WT plants, total RNA was isolated from leaf tissue using the RNeasy mini kit (Sigma-Aldrich), and then cDNA was synthesized using Verso cDNA synthesis kit (Thermo Scientific). qRT-PCR was performed using SYBR green kit (Thermo Scientific) according to the manufacturer's protocol. Due to the >89% sequence similarity observed between Arabidopsis and Camelina WSD1, the PCR primers used were designed based on the variable regions of mismatched sequences detected in WSD1 sequences after alignment. Based on the expression levels detected for WSD1 in Arabidopsis and Camelina transgenic plants, three lines are selected which exhibit low, moderate, and high levels of WSD1 expression, so we can easily correlate the expression patterns to the potential gene function.

### Cuticular Wax Loading and Composition Analysis

Cuticular waxes were extracted from Arabidopsis leaves (˜500 mg) and stems (˜200 mg) of 4-week old plants in chloroform (˜5 ml) for 30 seconds at room temperature following the method described by Seo et al. (Plant Cell (2011) 23:1138-115). The chloroform solvent extracts were then evaporated under N₂ steam, and subsequently the alkane standard, heptatriacontane (C₃₇H₇₆), was added as an internal standard for quantification of plant surface lipids. The lipid extracts were redissolved in a mixture of 25 μl acetonitrile and 25 μl of bis-N,N-trimethylsilyltrifluoroacetamide (BSTFA) and the mixtures were heated at 70° C. for 30 minutes to convert waxes into trimethylsilyl derivatives. The qualitative and quantitative analysis of cuticular waxes was conducted using gas chromatography with mass spectrometry (GC/MS) for identification, followed by gas chromatography with flame ionization detection (GC-FID) for quantitation.

### Chlorophyll Leaching Assays

The chlorophyll content in Arabidopsis leaves was determined as by Seo et al., 2011. Briefly, detached leaves of 4-week old plants grown in soil under growth chamber conditions were used. Approximately 300 mg of each leaf sample were incubated in ice for 30 min and then immersed in 30 ml of 80% ethanol in 50 ml conical tubes at room temperature. At the indicated time points after the initial immersion, ˜100 ul aliquots were removed from the solution and the amount of extracted chlorophylls were quantified by measuring absorbance at 647 and 664 nm using Evolution 60S UV-visible-spectrophotometer (Thermo Scientific). The amount of chlorophyll A, B and total chlorophylls was determined according to the formula described by (Arnon D I, 1949) of which ChlA (ug/ml)=12.7 (A663)−2.69 (A645); ChlB (ug/ml)=22.9 (A645)−4.68 (A663); total Chl (ug/ml)=20.2 (A645)+8.02 (A663). The difference in chlorophyll content measured at the indicated time points and the chlrophyll content measured after 24 h of leaf immersing in ethanol were expressed as percentages of the chlrophyll content measured after 24 h of leaf immersion in ethanol.

### Water Loss Assays

Four weeks old Arabidopsis plants grown in soil under growth chamber conditions were used for leaf water loss assay following the methods describe in Seo et al., 2011. Plants were dark-acclimated for ˜10 hours and then the detached rosetta leaves were soaked in water for 60 min in the dark. The leaves were dried and weighed at the indicated time points and the amount of water loss was presented as the percentage of the leaf weight at the indicated time points relative to the initial fresh weight. Three measurements were used and the average±standard error (SE) of the data was indicated.

### Scanning Electron Microscopy (SEM)

Cryogenic scanning electron microscopy (SEM, ZEIS, Inc.) was used to view epicuticular wax crystallization patterns. Inflorescence stem segments from tip to 3 cm and the fourth rosette leaves were collected from Arabidopsis wild-type (Col-0) and WSD1 overexpressing plants after 4 weeks of growth as described previously (Lü, S. et al. Plant J. (2009) 59, 553-564; Seo et al., 2011). Samples were fixed in 5% glutaraldehyde in 0.1 M Phosphate buffer (pH 6.8) for 2 hours at 4° C. before they washed in Phosphate buffer three times for 10 minutes each. The samples were subsequently dehydrated in a series of diluted ethanol (35%, 50%, 75%, 95%, 100%, 100%, 100%) for 15 minutes each. Then, the dehydrated samples were immersed in 1-2 ml of 50% and then 100% hexamethyldisilazane (HMDS) for 10 minutes each before they were air-dried in a desiccator overnight at room temperature. The air-dried samples were adhered to the cryo-holder using carbon tape and cryoadhesive were sputter coated with platinum and then transferred to the microscope cryo-stage for imaging.

### Statistical Analysis

The number of replicates (n) and the standard error (SE) are shown for most measurements. The data were analyzed with SAS version 9.1 using ANOVA (P<0.05) on the corresponding degrees of freedom (df), followed by Dunnett's procedure for pairwise comparisons of all treatments in transgenic plants compared to the nontransgenic WT control.

### Results

### Generation of Arabidopsis Transgenic Plants Overexpressing CaMV35S:: WSD1 Construct

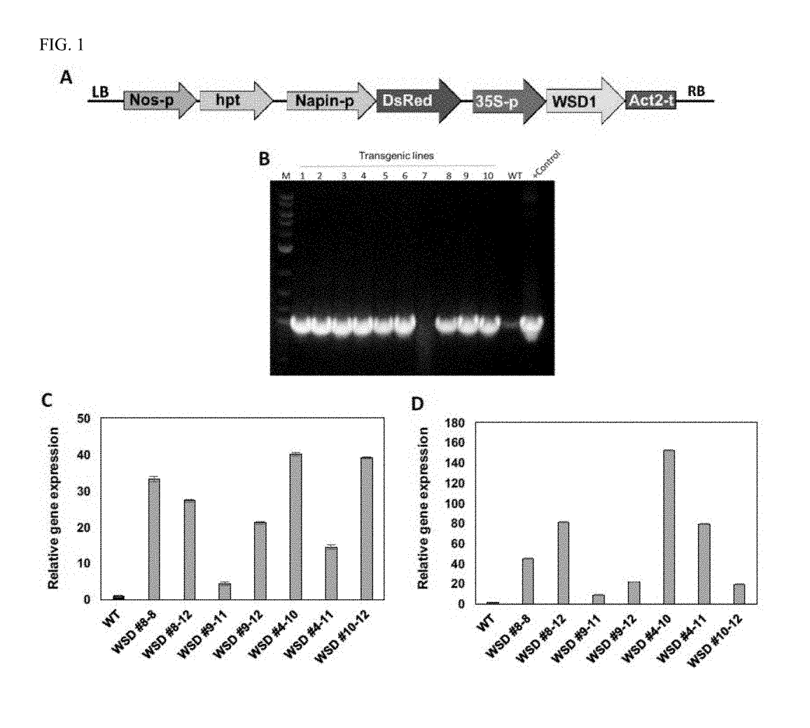

To study the physiological roles of the cuticular wax biosynthetic genes under abiotic stresses, transgenic Arabidopsis plants overexpressing AtWSD1 under the control of the constitutive CaMV35S promoter were generated (FIG. 1A). T1 seeds were screened for presence of the pCAMBIA-RedSeed vector carrying hygromycin phosphotransferase (hpt) by selecting the transformants on ½ strength MS medium containing 20 mg/L hygromycin-B antibiotic. The transgenic seedlings showing a hygromycin-resistance phenotype were grown to raise further generations.

To confirm the successful integration of CaMV35S::WSD1 construct into Arabidopsis genome, gDNAs were extracted from leaf tissues of the non-trasngenic WT as a control and 10 selected transgenic plants while pCAMBIA-RedSeed-WSD1 plasmid was used as a positive control. PCR-genotyping analysis was performed using specific PCR primers designed to detect the presence of CaMV35S promoter fragments. The analysis has confirmed that CaMV35S::WSD1 constructs were successfully integrated into the genomes of the transgenic plants (FIG. 1B). Further, to confirm the constitutive overexpression of WSD1, the shoots and roots of Arabidopsis plants grown on MS medium were subjected to qRT-PCR analyses (FIG. 1C-D). According to the figure, the relative expression of WSD1 transcripts was 5-40 folds and 10-160 folds higher in transgenic plants shoots and roots, respectively, as compared to non-transgenic WT plants. Altogether, both PCR and qRT-PCR results confirmed that WSD1 gene was successfully integrated and constitutively expressed at higher levels in transgenic lines.

Differential Expression of AtWSD1 under Stress Conditions

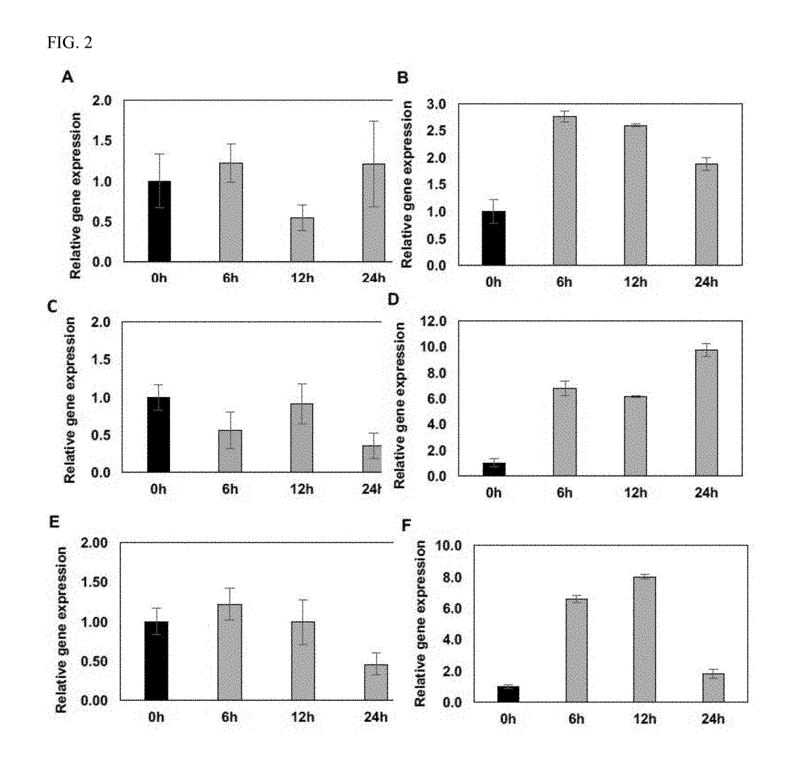

To determine whether the expression of AtWSD1 is induced by stress signaling hormones i.e. ABA and the stress conditions including mannitol and salt, qRT-PCR was performed to measure the relative expression of AtWSD1 in Arabidopsis shoot and root tissues (FIG. 2). qRT-PCR analyses indicated that the expression of AtWSD1 was induced rapidly upon exposure to mannitol, NaCl, and ABA treatments, particularly in shoots and no significant induction was detected in roots (FIG. 2). When treated with 100 mM mannitol, AtWSD1 transcript level was elevated more than 2.5 folds. It was peaked within 6h and then gradually declined (FIG. 2B). Further, the expression of AtWSD1 was also strongly up-regulated upto 10-folds by NaCl treatment (150 mM) within 6 h and was continuously elevated for up to 24 h (FIG. 2D). In addition, the expression of AtWSD1 was elevated upto 8 folds after exposure to ABA (1 uM), with its expression peaked within 12 h and then declined to normal levels within 24 h (FIG. 2F). These findings suggested that AtWSD1 probably contribute to regulate physiological roles in stress response aside from its wax synthase activity.

### AtWSD1 Overexpressing Transgenic Lines Exhibited Increased Abiotic Stress Stress Tolerance

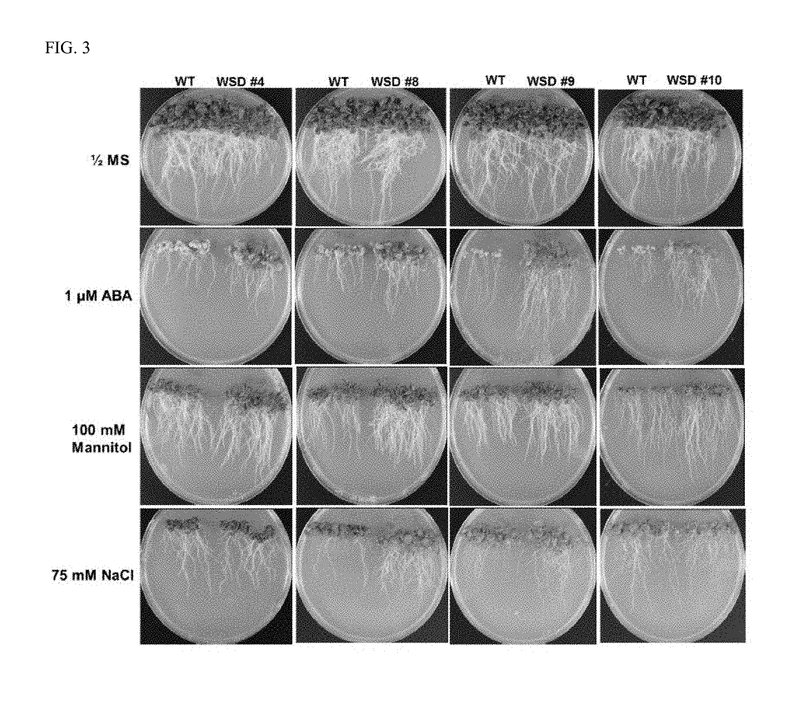

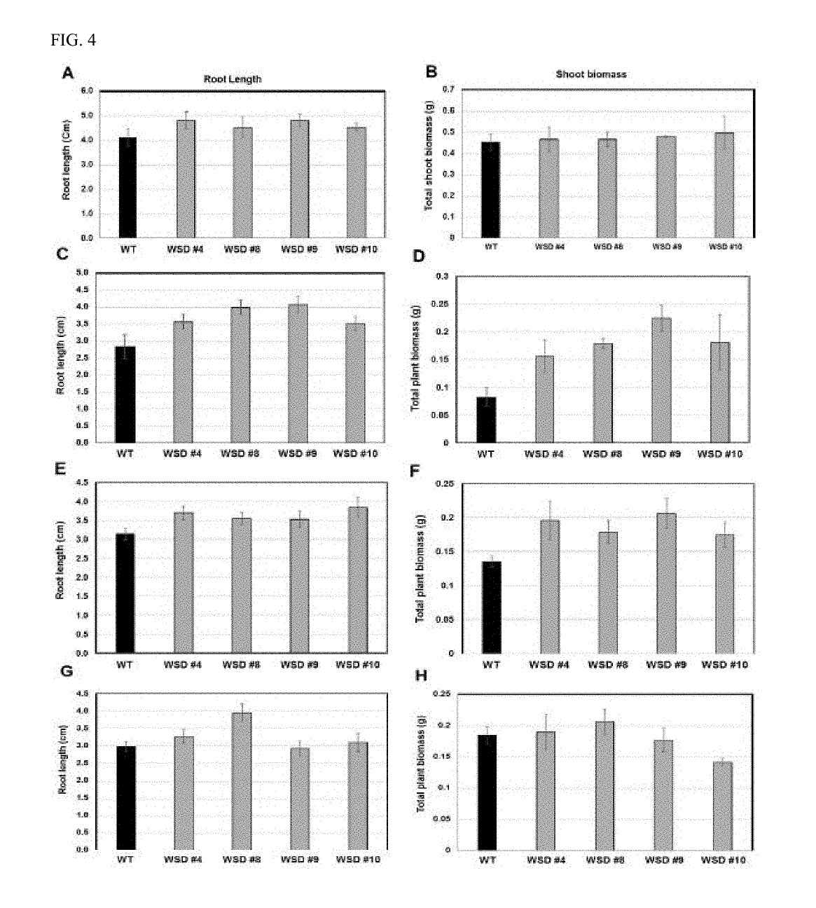

To analyze the impact of WSD1 overexpression in conferring tolerance to various stresses, including drought and salinity, AtWSD1 transgenics and WT seedlings were grown in half-strength MS (“½ MS”) medium supplemented with ABA (1 uM), mannitol (100 mM), and NaCl (75 mM). In the absence of stress agents, transgenic plants growth was similar to that in WT plants, with no difference in root lengths and total plant biomass (FIG. 3 and FIGS. 4A and 4B). When plated on ½ MS medium containing ABA, AtWSD1 transgenic lines grew better than the WT plants and exhibited longer roots and attained higher plant biomass. The root length was ˜1.5-fold and the plant biomass was ˜2.5-fold higher than that of the WT under ABA treatment (FIG. 3 and FIGS. 4C and 4D). Further, AtWSD1 transgenic plants treated with mannitol attained slightly but not significantly biggere root lengths, whereas the plant biomass was significantly increased (˜3-fold) in AtWSD1 transgenics compared to that in WT plants (FIG. 3 and FIGS. 4E and 4F). For AtWSD1 transgenic plants treated with salt (75 mM NaCl), the root length and shoot biomass slightly increased as compared to that in WT plants (FIG. 3 and FIGS. 4G and 4H). Collectively, the transcriptional induction of AtWSD1 under ABA and Mannitol and the resistant phenotype observed in AtWSD1 overexpressing lines under those treatments indicated the critical physiological roles AtWSD1 may play in drought, salt and ABA-mediated stress responses.

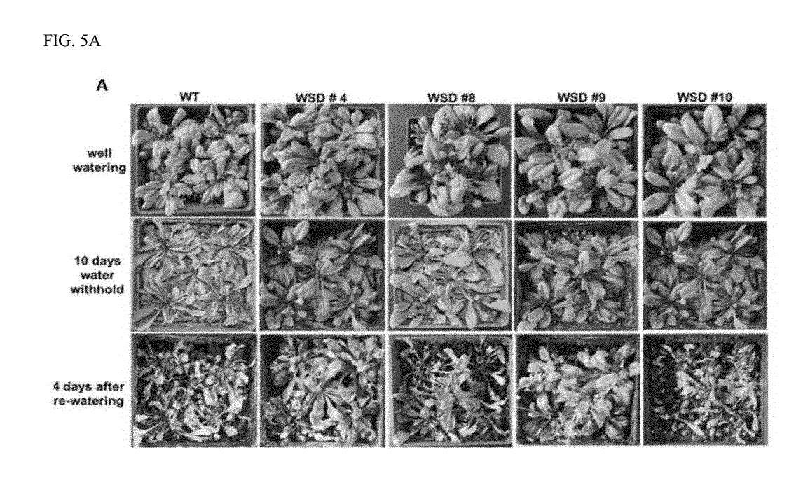

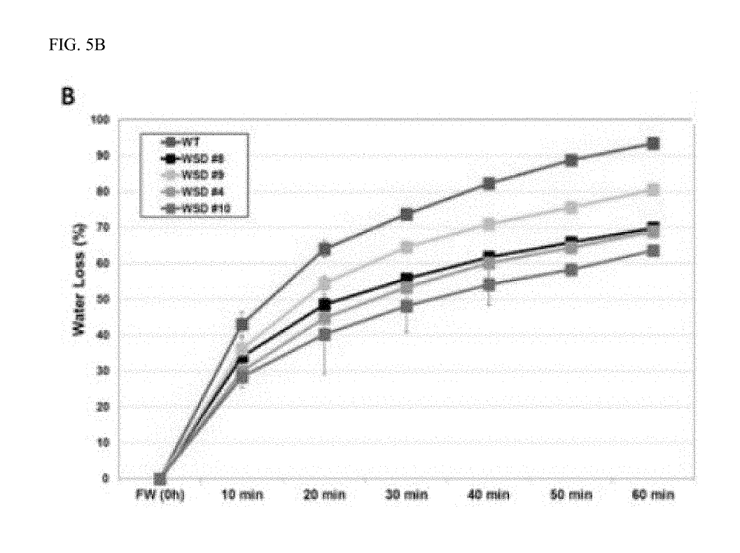

Since AtWSD1 was proven to be involved in cuticular wax biosynthesis and accumulation, we examined more directly whether increasing wax deposition by constitutively overexpressing AtWSD1 in Arabidopsis is linked with drought response in soil under greenhouse conditions. WT and AtWSD1 plants were subjected to drought by water withholding. The Arabidopsis AtWSD1 overexpressing lines and WT plants were grown under either normal or drought conditions and the leaves of 4-week-old plants were subject to water loss and chlorophyll leaching measurements. FIGS. 5A-5C show the results. The transgenic AtWSD1 plants exhibited a strong drought-tolerant phenotype after water was withheld for 12 days (FIG. 5A). The delayed wilting and drying in AtWSD1 transgenic plants was correlated with a reduction in water loss in leaf tissues, which indicate a reduction in transpiration (FIG. 5B). The results showed that the cuticular transpiration occurred more slowly in AtWSD1 transgenic leaves compared with WT. The transgenic leaf lost only ˜60% of its water content within 1 h, whereas, WT leaf lost ˜90% of its water content within the same period.

To support our findings that these changes along with alteration in epidermal properties have caused the drought and salt tolerance phenotypes, we performed chlorophyll leaching assays in Arabidopsis plants. Chlorophyll leaching was much reduced in leaves from AtWSD1 overexpressing transgenic plants as compared to WT leaves (FIG. 5C), possibly due to lower cuticle permeability. These findings showed that changing cuticular wax quantity or composition could be associated with reduced membrane permeability, and that AtWSD1-mediated cuticular wax biosynthesis is linked to drought tolerance/adaptation responses.

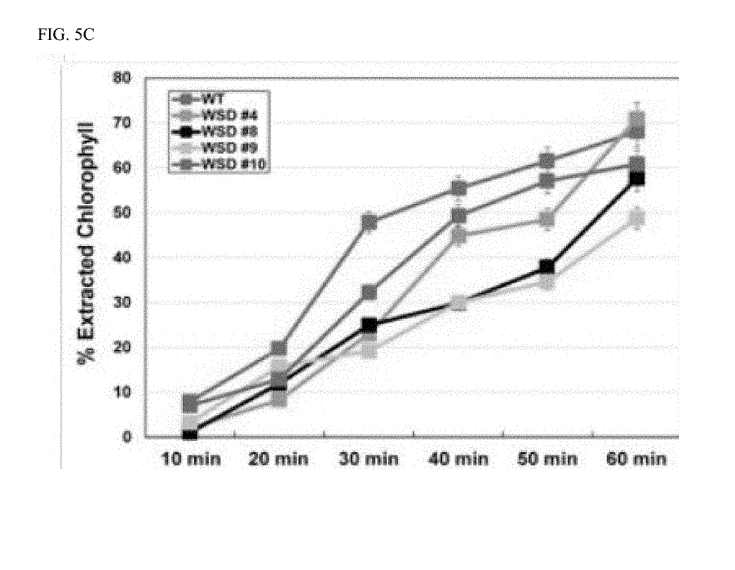

Next, we examined whether accumulation of more cuticular waxes via the overexpression of AtWSD1 can confer resistance to salinity stress. WT and transgenic plants were grown in soil under normal watering conditions. 4-week-old plants were subsequently watered with 100 mM NaCl-containing water for 20 days, and then allowed to recover for 10 days (FIG. 6). As shown in FIG. 6, the leaves of WT plants exhibited severe bleaching and cell death, whereas only a few leaves on transgenic plants exhibited slight wilting and after recovery, transgenic plants grew almost three times (biomass accumulatioin) better than WT plants.

### Epicuticular Wax Quantity and Composition is Altered in WSD1 Overexpressing Lines

To elucidate whether manipulating WSD1 expression can elevate the amount of waxes and/or alter their composition, GS/MS coupled with GC-FID and scanning electron microscopy (SEM) were used to profile the cuticular waxes deposited in Arabidopsis leaf and stem tissues and their content and composition were reported.

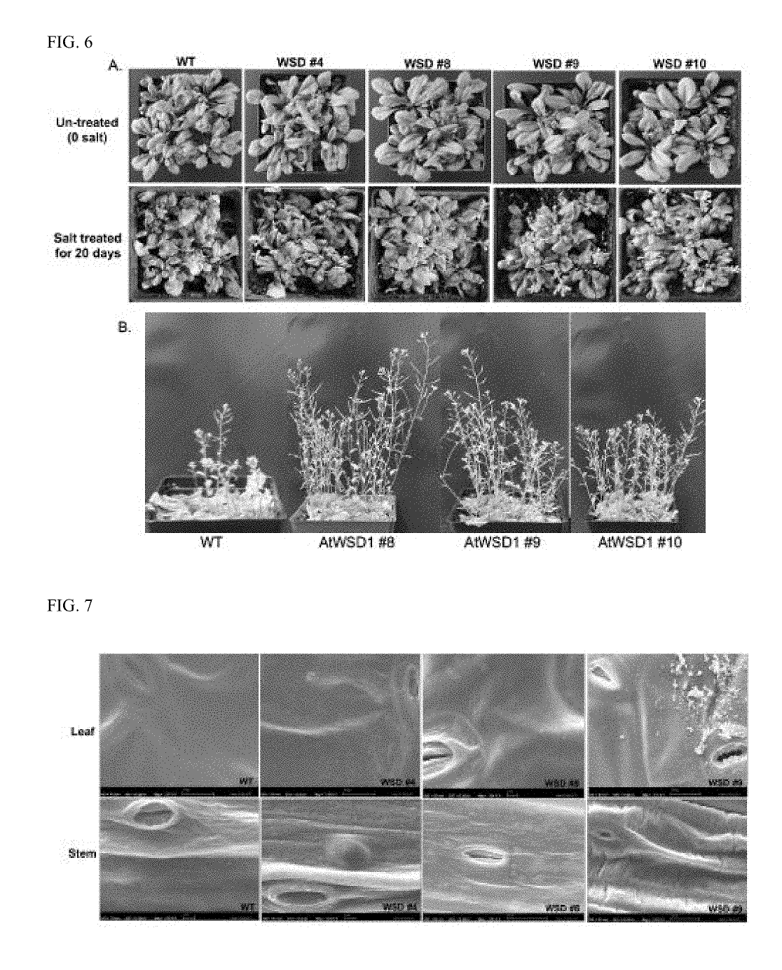

SEM imaging of Arabidopsis leaf and stem tissues indicated that the deposition of epicuticular wax crystals was increased on the transgenic leaf and stem surfaces relative to their amount in wild-type (WT) leaf and stem surfaces (FIG. 7).

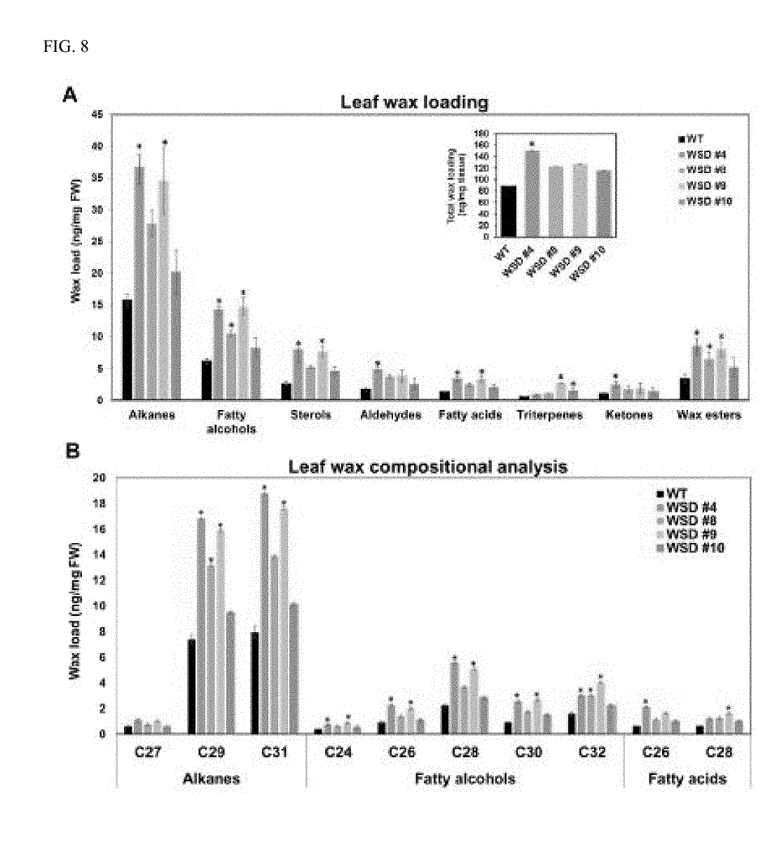

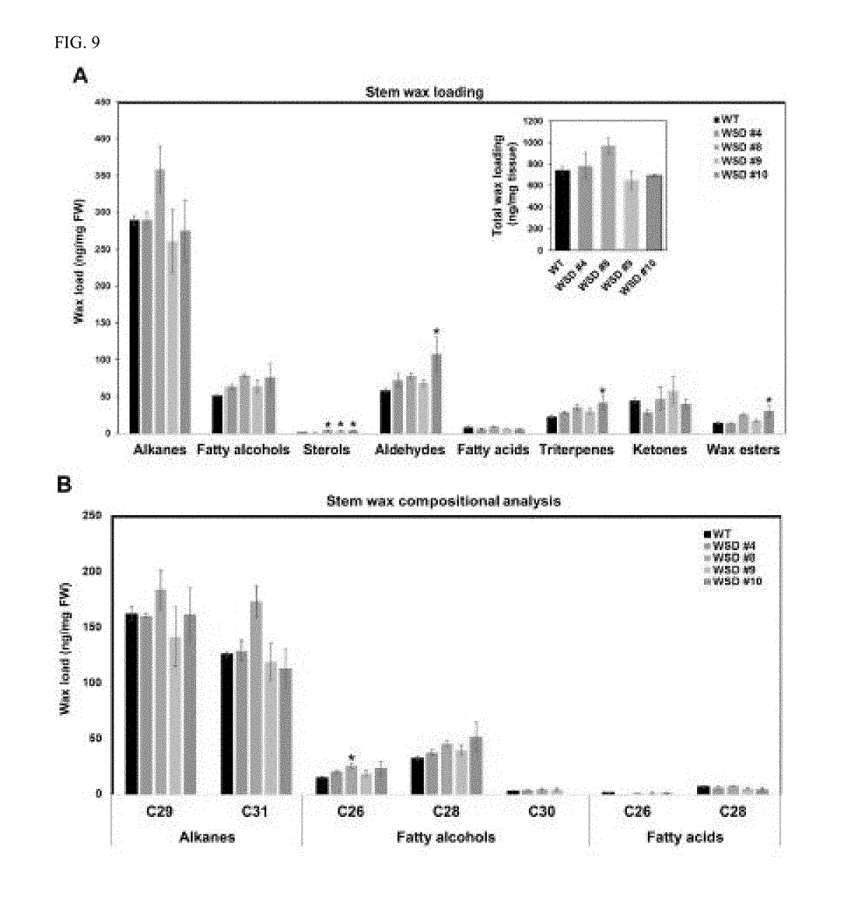

Further, measurement of cuticular wax content and composition in leaf and stem tissues of Arabidopsis WT and WSD1 transgenics by GC-FID and GC/MS, respectively, showed that the total wax load was increased by 40-70% in leaf (FIG. 8A) and by 15-30% in stem (FIG. 9A) tissues in WSD1 transgenics as compared to WT plants, supporting the observations from the SEM imaging.

Furthermore, overexpression of WSD1 altered the cuticular wax composition in Arabidopsis leaves (FIG. 8A). Leaf alkanes, fatty alcohols, sterols, aldehydes, wax esters, ketones, and fatty acids fractions were increased dramatically in WSD1 overexpressing lines as compared to WT (FIG. 8A). Alterations in the contents of alkanes and fatty alcohols were the most prominent changes. The levels of alkanes were elevated in WSD1 leaf (up to 77-130%) compared to their levels in WT, while fatty alcohols content increased by 72-141% in WSD1 leaf compare to that in WT leaf. Notably, most of the changes in the quantity of leaf wax in WSD1 lines could be attributed mostly to the 74-136% increases of the C29 and C31 alkanes, and 42-150% increases in C28 and C32 fatty alcohols (FIG. 8B).

Unlike in Arabidopsis leaf, the impact of WSD1 overexpression on the cuticular wax amount and composition in stem tissues was less obvious as the total wax loading did not change in the transgenic plants, and the levels of alkanes, fatty alcohols, ketones, and fatty acids remained unaffected. There were significant increases in the levels of sterols, which originally accumulated in less abundance, and only an individual line (WSD #10) did exhibit increased accumulation of aldehydes, triterpenes, and wax esters (FIG. 9).

### Example 2

### Transforming Various Other Crops with the WSD1 Constructs

### Agrobacterium-Mediated Transformation of Brassica Napus (Canola)

Plant material: Mature seeds are surface sterilized in 10% commercial bleach for 30 min with gentle shaking and washed three times with sterile distilled water.

Culture initiation and transformation: Seeds are plated on germination medium (MS basal medium supplemented with 30 g/l sucrose) and incubated at 24° C. with a 16-h photoperiod at a light intensity of 60-80 μE/m²/s for 4-5 d. For transformation, cotyledons with ˜2 mm of the petiole at the base are excised from the resulting seedlings, immersed in Agrobacterium tumefacians strain EHA101 suspension (grown from a single colony in 5 ml of minimal medium supplemented with appropriate antibiotics at 28° C. for 48 h) for 1 s and immediately embedded to a depth of ˜2 mm in a co-cultivation medium (MS basal medium with 30 g/l sucrose and 20 μM benzyladenine). The inoculated cotyledons are incubated under the same growth conditions for 48 h.

Plant regeneration and selection: After co-cultivation, cotyledons are transferred on to a regeneration medium comprising MS medium supplemented with 30 g/l sucrose and 20 μM benzyladenine, 300 mg/l timentinin and 20 mg/l kanamycin sulfate. After 2-3 weeks, regenerated shoots are cut and maintained on MS medium for shoot elongation containing 30 g/l sucrose, 300 mg/l timentin, and 20 mg/l kanamycin sulfate. The elongated shoots are transferred to a rooting medium comprising MS basal medium supplemented with 30 g/l sucrose, 2 mg/l indole butyric acid (IBA) and 500 mg/L carbenicillin. After root formation, plants are transferred to soil and grown to seed maturity under growth chamber or greenhouse conditions.

### Agrobacterium-Mediated Transformation of Soybean

The vectors are used for Agrobacterium-mediated transformation of soybean following a previously described procedure (Ko et al., 2006, Agrobacterium Protocols Wang K., ed., Vol. 1, pp 397-405, Humana Press).

Plant material: Immature seeds from soybean plants grown under greenhouse or field conditions are used as an explant source. Young pods are harvested and surface sterilized with 70% 2-propanol for 30 sec and 25% Clorox for 20 min followed by three washes with sterile distilled water.

Culture transformation and selection: Under aseptic conditions, immature seeds are removed from the pods and the cotyledons are separated from the seed coat followed by incubation in A. tumefaciens culture (grown from a single colony at 28° C., overnight) in co-cultivation medium (MS salts and B5 vitamins) supplemented with 30 g/l sucrose, 40 mg/l 2,4-D and 40 mg/l acetosyringone for 60 min. Infected explants are plated abaxial side up on agar-solidified co-cultivation medium and incubated at 25° C., in the dark for 4 d.

For selection of transformed tissues, cotyledons washed with 500 mg/l cephotaxine are placed abaxial side up on a medium for induction of somatic embryo formation (Gelrite-solidified MS medium medium containing 30 g/l sucrose, 40 mg/l 2,4-D, 500 mg/l cefotaxime, and 10 mg/l hygromycin) and incubated at 25° C., under a 23-h photoperiod (10-20 μE/m2/s) for 2 weeks. After another two weeks of growth under the same conditions in the presence of 25 mg/l hygromycin, the antibiotic-resistant somatic embryos are transferred on MS medium for embryo maturation supplemented with 60 g/l maltose, 500 mg/l cefotaxime, and 10 mg/l hygromycin and grown under the same conditions for 8 weeks with 2-week subculture intervals.

Plant regeneration and selection: The resulting cotyledonary stage embryos are desiccated at 25° C., under a 23-h photoperiod (60-80 μE/m2/s) for 5-7 d followed by culture on MS regeneration medium containing 30 g/l sucrose and 500 mg/l cefotaxime for 4-6 weeks for shoot and root development. When the plants are 5-10 cm tall, they are transferred to soil and grown in a greenhouse after acclimatization for 7 d.

### Agrobacterium-Mediated Transformation of Rice

The vectors provided in the invention can be used for Agrobacterium-mediated transformation of rice following a previously described procedure (Herve and Kayano, 2006, Agrobacterium Protocols Wang K., ed., Vol. 1, pp 213-222, Humana Press).

Plant material: Mature seeds from japonica rice varieties grown in a greenhouse are used as an explant source.

Culture transformation and selection: Dehusked seeds are surface sterilized with 70% ethanol for 1 min and 3% sodium hypochlorite for 30 min followed by six washes with sterile distilled water. Seeds are plated embryo side up on an induction medium (Gelrite-solidified N6 basal medium supplemented with 300 mg/l casamino acids, 2.88 g/l proline, 30 g/l sucrose and 2 mg/l 2,4-D) and incubated at 32° C., under continuous light for 5 d. Germinated seeds with swelling of the scutellum are infected with A. tumefaciens strain LBA4404 (culture from 3-d-old plates resuspended in N6 medium supplemented with 100 μM acetosyringone, 68.5 g/l sucrose and 36 g/l glucose) at room temperature for 2 min followed by transfer on to a co-cultivation medium (N6 Gelrite-solidified medium containing 300 mg/l casamino acids, 30 g/l sucrose, 10 g/l glucose, 2 mg/l 2,4-D and 100 μM acetosyringone) and incubation at 25° C., in the dark for 3 d.

For selection of transformed embryogenic tissues, whole seedlings washed with 250 mg/l cephotaxine are transferred on to N6 agar-solidified medium containing 300 mg/l casamino acids, 2.88 g/l proline, 30 g/l sucrose, 2 mg/l 2,4-D, 100 mg/l cefotaxime, 100 mg/l vancomycin and 35 mg/l G418 disulfate). Cultures are incubated at 32° C., under continuous light for 2-3 weeks.

Plant regeneration and selection: Resistant proliferating calluses are transferred on to agar-solidified N6 medium containing 300 mg/l casamino acids, 500 mg/l proline, 30 g/l sucrose, 1 mg/l NAA, 5 mg/l ABA, 2 mg/l kinetin, 100 mg/l cefotaxime, 100 mg/l vancomycin and 20 mg/l G418 disulfate. After one week of growth at 32° C., under continuous light, the surviving calluses are transferred on to MS medium (solidified with 10 g/l agarose) supplemented with 2 g/l casamino acids, 30 g/l sucrose, 30 g/l sorbitol, 0.02 mg/l NAA, 2 mg/l kinetin, 100 mg/l cefotaxime, 100 mg/l vancomycin and 20 mg/l G418 disulfate and incubated under the same conditions for another week followed by a transfer on to the same medium with 7 g/l agarose. After 2 weeks, the emerging shoots are transferred on to Gelrite-solidified MS hormone-free medium containing 30 g/l sucrose and grown under continuous light for 1-2 weeks to promote shoot and root development. When the regenerated plants are 8-10 cm tall, they can be transferred to soil and grown under greenhouse conditions. After about 10-16 weeks, transgenic seeds are harvested.

Indica rice varieties are transformed with Agrobacterium following a similar procedure (Datta and Datta, 2006, Agrobacterium Protocols Wang K., ed., Vol. 1, pp 201-212, Humana Press).

### Transformation of Wheat by Microprojectile Bombardment

The gene constructs provided in the invention can be used for wheat transformation by microprojectile bombardment following a previously described protocol (Weeks et al., 1993, Plant Physiol. 102: 1077-1084).

Plant material: Plants from the spring wheat cultivar Bobwhite are grown at 18-20° C. day and 14-16° C. night temperatures under a 16 h photoperiod. Spikes are collected 10-12 weeks after sowing (12-16 days post anthesis). Individual caryopses at the early-medium milk stage are sterilized with 70% ethanol for 5 min and 20% sodium hypochlorite for 15 min followed by three washes with sterile water.

Culture initiation, transformation and selection: Immature zygotic embryos (0.5-1.5 mm) are dissected under aseptic conditions, placed scutellum side up on a culture induction medium (Phytagel-solidified MS medium containing 20 g/l sucrose and 1.5 mg/l 2,4-D) and incubated at 27° C., in the light (43 μmol/m²/s) for 3-5 d.

For microprojectile bombardment, embryo-derived calluses are plated on the culture initiation medium supplemented with 0.4 M sorbitol 4 hours before gene transfer. Plasmid DNA containing the expression cassettes is precipitated onto 0.6-μm gold particles and delivered to the explants as described in Weeks et al., 1993.

The bombarded expants are transferred to callus selection medium (the culture initiation medium described above containing 1-2 mg/l bialaphos) and subcultured every 2 weeks.

Plant regeneration and selection: After one-two selection cycles, cultures are transferred on to MS regeneration medium supplemented with 0.5 mg/l dicamba and 2 mg/l bialaphos. For root formation, the resulting bialaphos-resistant shoots are transferred to hormone-free half-strenght MS medium. Plants with well-developed roots are transferred to soil and acclimated to lower humidity at 21° C. with a 16-h photoperiod (300 mol/m²/s) for about 2 weeks prior to transfer to a greenhouse.

In conclusion, this disclosure show that WSD1 is implicated in wax synthesis in leaves and stem, and has a subsequent role in providing tolerance to abiotic stresses, including drought, salinity, and ABA. Additionally, the increased wax deposition on leaves and stem surfaces as a result of WSD1 overexpression may also provide a physical barrier and may provide tolerance to pathogens and insect damage in plants. Transgenic plants overxpressing WSD1 thus have strong potential to be utilized in developing food, forage, feed, and biofuels crops with increased tolerance to various environmental stresses and producing better crop yield under extreme environments.

The compositions and methods disclosed herein include(s) at least the following aspects:

Aspect 1. A method of increasing stress-tolerance in a plant or in a part, cell, or propagation material thereof, comprising: expressing a transgenic expression cassette in a plant or in a tissue, organ, part, cell or propagation material thereof, wherein the transgenic expression cassette comprises a nucleic acid sequence encoding a wax synthase/acyl-CoA:diacylglycerol acyltransferase (WSD1) operatively linked to a plant-expressible promoter; and selecting the plant or the tissue, organ, part, cell or propagation material thereof in which expression of WSD1 is increased, cuticular wax content is increased in leaves or stem, the water loss from leaves is reduced, or chlorophyll leaching is reduced in comparison with a plant of the same species, or a tissue, organ part, cell or propagation material thereof, that is not expressing the transgenic expression cassette.

Aspect 2. A method of producing a transgenic plant having increased stress-tolerance, the method comprises transforming a plant cell with a transgenic expression cassette comprising a nucleic acid sequence encoding a wax synthase/acyl-CoA:diacylglycerol acyltransferase (WSD1) operatively linked to a plant-expressible promoter; growing a plant from the transformed plant cell until the plant produces seed; and selecting a seed from a plant in which the expression of WSD1 is increased, cuticular wax content is increased in leaves or stem, the water loss from leaves is reduced, or chlorophyll leaching is reduced relative to a plant of the same species not comprising the transgenic expression cassette.

Aspect 3. The method of aspect 1 or 2, wherein the WSD1 polypeptide is from a plant selected from Arabidopsis , Tropaeolum, Brassica, soybean (Glycine max), Linum, Helianthus, Camelina, Arachis, Ricinus, Cuphea, Crambe, Avocado (Persea americana), wheat (Triticum), rice (Oryza), maize, sorghum, barley, or Gossipium; preferably Arabidopsis.

Aspect 4. The method of any one of aspects 1 to 3, wherein the plant is selected from Amaranthaceae, Asteraceae, Brassicaceae, Carophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanaceae, Sterculiaceae, Tetragoniaceae, Theaceae, Umbelliferae, or Poaceae; preferably Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, or Populus; more preferably Arabidopsis thaliana, Borago species (spp.), Canola, Ricinus spp., Theobroma spp., Zea spp., Gossypium spp, Crambe spp., Cuphea spp., Linum spp., Lesquerella spp., Limnanthes spp., Linola, Tropaeolum spp., Oenothera spp., Olea spp., Elaeis spp., Arachis spp., rapeseed, Carthamus spp., Glycine spp., Sojaspp., Helianthus spp., Nicotiana spp., Vernonia spp., Triticum spp., Hordeum spp., Oryza spp., Avena spp., Sorghum spp., pearl millet, foxtail (Setaria viridis), Secale spp., Brassicaceae, or Poaceae spp.

Aspect 5. The method of any one of aspects 1 to 4, wherein increased WSD1 expression is determined as a ratio of WSD1 gene expression in the transgenic plant or a tissue, organ part, cell or propagation material thereof compared to WSD1 gene expression in a plant of the same species or a tissue, organ part, cell or propagation material thereof not comprising the transgenic expression cassette.

Aspect 6. The method of any one of aspects 1 to 5, wherein the ratio is at least 2, or at least 5.

Aspect 7. The method of any one of aspects 1 to 6, wherein water loss at one hour from leaves of the plant is decreased by at least 5%, at least 10%, at least 15%, at least 20% or at least 30%, compared to water loss at one hour from leaves of a plant of the same species not comprising the transgenic expression cassette.

Aspect 8. The method of any one of aspects 1 to 7, wherein chlorophyll leaching at one hour from leaves of the plant is decreased by at least 5%, at least 10%, at least 15%, or at least 20%, or at least 30% compared to chlorophyll leaching at one hour from leaves of a plant of the same species not comprising the transgenic expression cassette.

Aspect 9. The method of any one of aspects 1 to 8, wherein cuticular wax content is increased in leaves of the plant by at least 5%, 10%, 20%, 30%, 40%, 50, %, 60%, or 70% compared to a plant of the same species not comprising the transgenic expression cassette.

Aspect 10. The method of any one of aspects 1 to 8, wherein cuticular wax content is increased in stems of the plant by at least 5%, 10%, 15%, 20, %, 25%, 30%, 40%, 50% compared to a plant of the same species not comprising the transgenic

Aspect 11. The method of any onexpression cassette. e of aspects 1 to 10, wherein WSD1 has a sequence comprising SEQ ID NO:1.

Aspect 12. The method of any one of aspects 1 to 11, wherein the plant-expressible promoter is a constitutive promoter or a light regulated promoter.

Aspect 13. The method of aspect 12, wherein the plant-expressible promoter is a Cauliflower mosaic virus 35S (CaMV35S) promoter.

Aspect 14. A trangenic plant or a tissue, organ, part, cell, or propagation material thereof made by the method of any one of aspects 1 to 13.

Aspect 15. The transgenic plant or the tissue, organ, part, cell, or propagation material thereof of aspect 14 which is a seed.

Aspect 16. A transgenic expression cassette comprising a polynucleotide encoding a wax synthase/acyl-CoA:diacylglycerol acyltransferase (WSD1) operatively linked to a plant-expressible promoter.

Aspect 17. The expression cassette of aspect 16, wherein the WSD1 polypeptide is from a plant selected from Arabidopsis , Tropaeolum, Brassica, soybean (Glycine max), Linum, Helianthus, Camelina, Arachis, Ricinus, Cuphea, Crambe, Avocado (Persea americana), wheat (Triticum), rice (Oryza), maize, foxtail (Setaria viridis), sorghum, pearl millet, barley, or Gossipium; preferably Arabidopsis.

Aspect 18. The expression cassette of aspect 16 or 17, wherein WSD1 has a sequence comprising SEQ ID NO:1.

Aspect 19. The expression cassette of any one of aspects 16 to 18 wherein the plant-expressible promoter is a constitutive promoter or a light regulated promote, preferably a Cauliflower mosaic virus 35S (CaMV35S) promoter.

Aspect 20. The expression cassette of any one of aspects 16 to 19 wherein the polynucleotide is further operatively linked to the ACT2 terminator.

Aspect 21. The expression cassette of any one of aspects 16 to 20 wherein the polynucleotide is cloned within the vector pCambia-RedSeed.

Aspect 22. A transgenic plant comprising the transgenic expression cassette of any one of aspects 16 to 21.

Aspect 23. The transgenic plant of aspect 220, characterized by increased WSD1 expression, determined as a ratio of WSD1 gene expression in the transgenic plant or a tissue, organ part, cell or propagation material thereof compared to WSD1 gene expression in a plant of the same species or a tissue, organ part, cell or propagation material thereof not comprising the transgenic expression cassette, wherein the ratio is at least 2, or at least 5; cuticular wax content is increased in leaves of the plant by at least 5%, 10%, 20%, 30%, 40%, 50, %, 60%, or 70% compared to a plant of the same species not comprising the transgenic expression cassette; cuticular wax content is increased in stems of the plant by at least 5%, 10%, 15%, 20%, 25%, 30%, 40%, or 50% compared to a plant of the same species not comprising the transgenic expression cassett; water loss from leaves reduced by at least 5%, at least 10%, at least 15%, at least 20%, or at least 30% compared to a plant of the same species not comprising the transgenic expression cassette; or chlorophyll leaching from leaves reduced by at least 5%, at least 10%, at least 15%, at least 20%, or at least 30% compared to a plant of the same species not comprising the transgenic expression cassette.

Aspect 24. The transgenic plant of aspect 22 or 23, wherein the plant is selected from Amaranthaceae, Asteraceae, Brassicaceae, Carophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanaceae, Sterculiaceae, Tetragoniaceae, Theaceae, Umbelliferae, or Poaceae; preferably Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, or Populus; more preferably Arabidopsis thaliana, Borago species (spp.), Canola, Ricinus spp., Theobroma spp., Zea spp., Gossypium spp, Crambe spp., Cuphea spp., Linum spp., Lesquerella spp., Limnanthes spp., Linola, Tropaeolum spp., Oenothera spp., Olea spp., Elaeis spp., Arachis spp., rapeseed, Carthamus spp., Glycine spp., Soja spp., Helianthus spp., Nicotiana spp., Vernonia spp., Triticum spp., Hordeum spp., Oryza spp., Avena spp., Sorghum spp., pearl millet, foxtail (Setaria viridis), Secale spp., Brassicaceae, or Poaceae spp.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A method of increasing stress-tolerance in a plant or in a part, cell, or propagation material thereof, wherein the stress is drought, high salinity, high temperature, or a biotic stress, comprising:
expressing a transgenic expression cassette in a plant or in a tissue, organ, part, cell or propagation material thereof, wherein the transgenic expression cassette comprises a nucleic acid sequence encoding a wax synthase/acyl-CoA:diacylglycerol acyltransferase (WSD1) operatively linked to a plant-expressible promoter; and
selecting the plant or the tissue, organ, part, cell or propagation material thereof in which expression of WSD1 is increased, cuticular wax content is increased in leaves or stem, water loss from leaves is reduced, or chlorophyll leaching is reduced in comparison with a plant of the same species, or a tissue, organ part, cell or propagation material thereof, that is not expressing the transgenic expression cassette.

2. A method of producing a transgenic plant having increased stress-tolerance, wherein the stress is drought, high salinity, high temperature, or a biotic stress, the method comprises
transforming a plant cell with a transgenic expression cassette comprising a nucleic acid sequence encoding a wax synthase/acyl-CoA: diacylglycerol acyltransferase (WSD1) operatively linked to a plant-expressible promoter;
growing a plant from the transformed plant cell until the plant produces seed; and
selecting a seed from a plant in which expression of WSD1 is increased, cuticular wax content is increased in leaves or stem, water loss from leaves is reduced, or chlorophyll leaching is reduced relative to a plant of the same species not comprising the transgenic expression cassette.

3. The method of claim 1, wherein the WSD1 polypeptide is from a plant selected from Arabidopsis, Tropaeolum, Brassica, soybean (Glycine max), Linum, Helianthus, Camelina, Arachis, Ricinus, Cuphea, Crambe, Avocado (Persea americana), wheat (Triticum), rice (Oryza), maize, foxtail (Setaria viridis) sorghum, pearl millet, barley, or Gossypium.

4. The method of claim 1, wherein the plant is selected from Amaranthaceae, Asteraceae, Brassicaceae, Carophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanaceae, Sterculiaceae, Tetragoniaceae, Theaceae, Umbelliferae, or Poaceae.

5. The method of claim 1, wherein increased WSD1 expression is determined as a ratio of WSD1 gene expression in the transgenic plant or a tissue, organ part, cell or propagation material thereof compared to WSD1 gene expression in a plant of the same species or a tissue, organ part, cell or propagation material thereof not comprising the transgenic expression cassette, wherein the ratio is at least 2.

6. The method of claim 1, wherein water loss at one hour from leaves of the plant is decreased by at least 5% compared to water loss at one hour from leaves of a plant of the same species not comprising the transgenic expression cassette;
chlorophyll leaching at one hour from leaves of the plant is decreased by at least 5% compared to chlorophyll leaching at one hour from leaves of a plant of the same species not comprising the transgenic expression cassette;
cuticular wax content is increased in leaves of the plant by at least 5% compared to a plant of the same species not comprising the transgenic expression cassette; or
cuticular wax content is increased in stems of the plant by at least 5%compared to a plant of the same species not comprising the transgenic expression cassette.

7. The method of claim 1, wherein WSD1 has a sequence comprising SEQ ID NO:1.

8. The method of claim 1, wherein the plant-expressible promoter is a constitutive promoter or a light regulated promoter.

9. A transgenic plant or a tissue, organ, part, cell, or propagation material thereof made by the method of claim 1.

10. The transgenic plant or the tissue, organ, part, cell, or propagation material thereof of claim 9 which is a seed.

11. A transgenic expression cassette comprising a polynucleotide encoding a wax synthase/acyl-CoA:diacylglycerol acyltransferase (WSD1) operatively linked to a plant-expressible promoter.

12. The expression cassette of claim 11, wherein the WSD1 polypeptide is from a plant selected from Arabidopsis, Tropaeolum, Brassica, soybean (Glycine max), Linum, Helianthus, Camelina, Arachis, Ricinus, Cuphea, Crambe, Avocado (Persea americana), wheat (Triticum), rice (Oryza), maize, foxtail (Setaria viridis) sorghum, pearl millet, barley, or Gossypium.

13. The expression cassette of claim 11, wherein WSD1 has a sequence comprising SEQ ID NO:1.

14. The expression cassette of claim 11 wherein the plant-expressible promoter is a constitutive promoter or a light regulated promoter.

15. The expression cassette of claim 11 wherein the polynucleotide is further operatively linked to the ACT2 terminator.

16. The expression cassette of claim 11 wherein the polynucleotide is cloned within the vector pCambia-RedSeed.

17. A transgenic plant comprising the transgenic expression cassette of claim 11.

18. The transgenic plant of claim 17, characterized by
increased WSD1 expression, determined as a ratio of WSD1 gene expression in the transgenic plant or a tissue, organ part, cell or propagation material thereof compared to WSD1 gene expression in a plant of the same species or a tissue, organ part, cell or propagation material thereof not comprising the transgenic expression cassette, wherein the ratio is at least 2;
cuticular wax content is increased in leaves of the plant by at least 5% compared to a plant of the same species not comprising the transgenic expression cassette;
cuticular wax content is increased in stems of the plant by at least 5% compared to a plant of the same species not comprising the transgenic expression cassette;
water loss from leaves reduced by at least 5% compared to a plant of the same species not comprising the transgenic expression cassette; or
chlorophyll leaching from leaves reduced by at least 5% compared to a plant of the same species not comprising the transgenic expression cassette.

19. The transgenic plant of claim 22, wherein the plant is selected from Amaranthaceae, Asteraceae, Brassicaceae, Carophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanaceae, Sterculiaceae, Tetragoniaceae, Theaceae, Umbelliferae, or Poaceae.
